(19) United States
(12) Patent Application Publication
(10) Pub. No.: US 2009/0130101 A1
(43) Pub. Date: May 21, 2009

(54) ANTI-CANCER THERAPY WITH AN EXTRACT OF SCUTELLARIA BARBATA

(75) Inventor: Isaac Cohen, Piedmont, CA (US)

Correspondence Address: WILSON SONSINI GOODRICH & ROSATI, 650 PAGE MILL ROAD, PALO ALTO, CA, 94304-1050, US

(73) Assignee: BIONOVO, INC., Emeryville, CA (US)

(21) Appl. No.: 12/274,251

(22) Filed: Nov. 19, 2008

Related U.S. Application Data
(60) Provisional application No. 60/989,069, filed on Nov. 19, 2007.

(51) Int. Cl. A61K 39/395; A61K 36/539; A61P 35/00

(52) U.S. Cl. 424/133.1; 424/725; 424/155.1

(57) ABSTRACT

Methods of treating cancer with a combination of an extract of Scutellaria barbata D. Don and at least one additional anticancer chemotherapeutic agent are provided. Also provided are kits comprising an extract of Scutellaria barbata D. Don and at least one additional anticancer chemotherapeutic agent.

This application claims benefit of priority under 35 U.S.C. § 119(e) from provisional patent application 60/989,069, filed Nov. 19, 2007, which is incorporated herein by reference in its entirety.

### BACKGROUND OF THE INVENTION

While advances in early detection and adjuvant therapy for breast cancer have had a favorable impact on patient survival in general, patients who develop advanced metastatic breast cancer are generally likely to face a less favorable prognosis. Commonly used hormonal and chemotherapeutic agents can lead to transient regression of tumors and can also palliate symptoms related to cancer. However, these treatments are often accompanied by toxicities and intolerable side effects and eventually become ineffective in controlling advanced stage breast cancer and its symptoms. Improvements in survival are modest, even with newer targeted biological agents. Moreover, in most metastatic cancers resistance to available conventional treatment ultimately develops or excessive side effects are seen with conventional therapies.

It is interesting to note that greater than 60% of all chemotherapeutic agents used in the treatment of breast cancer are derived from natural substances (Newman 2003). A fairly recent example is the development of taxanes from the Pacific yew tree, Taxus brevifolia. Throughout the world, it is estimated that approximately 80% of the world population still relies on botanical medicine as the primary source of therapy. In the West, botanical medicine is considered a popular form of complementary and alternative medicine among patients diagnosed with cancer. However, few clinical trials have been conducted to firmly assess the safety and efficacy of botanical agents for the treatment of breast cancer, despite anecdotal case reports of cures and clinical efficacy in women who have relied solely on botanical medicine for treatment. It has previously been shown that the aqueous extract of Scutellaria Barbata can lead to growth inhibition of breast cancer cell lines in vitro (“Antiproliferative activity of Chinese medicinal herbs on breast cancer cells in vitro,” Anticancer Res., 22(6C):3843-52 (2002)). BZL101, a concentrated aqueous extract of Scutellaria Barbata, was evaluated for antiproliferative activity on five breast cancer cell lines (SK-BR-3, MCF7, MDA-MB-231, BT-474, and MCNeuA). These cell lines represent important prognostic phenotypes of breast cancer expressing a range of estrogen and HER2 receptors. BZL101, tested at a 1:10 dilution (15 μg/ml), demonstrated >50% growth inhibition on four of the five cell lines (Campbell, 2002). BZL101 showed >50% growth inhibition on a panel of lung, prostate and pancreatic cancer cell lines. BZL101 at the same dose did not cause >25% of growth inhibition on normal human mammary cells (HuMEC), demonstrating selectivity to cancer cells (Table 1). More so, BZL101 had a mild mitogenic effect on normal human lymphocytes. In cell cycle analysis, BZL101 caused an S phase burst and G1 arrest. BZL101 also attenuated mitochondrial membrane potential causing caspase-independent high molecular grade (HMG) apoptosis.

### SUMMARY OF INVENTION

The inventor has recognized a need for improved methods of treating various types of cancer, especially ER⁻ (e.g. ERα⁻ and/or ERβ⁻) breast cancer. Various embodiments of the invention provided herein meet the foregoing need and provide related advantages as well.

Thus, some embodiments described herein provide a method of treating metastatic breast cancer having low or no expression of nuclear estrogen receptor (ER) in a patient, comprising: (a) determining that the expression of ER in the cancer is below a predetermined threshold; and (b) provided that the expression of ER is below the predetermined threshold, administering to the patient a therapeutically effective amount of an extract of Scutellaria Barbata D. Don.

Some embodiments described herein provide a method of treating an estrogen receptor modulator treatment-refractory metastatic breast cancer, comprising: (a) determining that the tumor is refractory to treatment with an estrogen receptor modulator; and (b) provided that the tumor is refractory to treatment with an estrogen receptor modulator, administering to the patient a therapeutically effective amount of an extract of Scutellaria Barbata D. Don. In some embodiments, the method further comprises determining that the tumor expresses nuclear estrogen receptor (ER) above a predetermined threshold. In some embodiments, the estrogen receptor modulator is tamoxifen or raloxifene. In some embodiments, the method further comprises determining that the tumor is refractory to treatment with an estrogen receptor modulator comprises administering the estrogen receptor modulator to a patient and determining that treatment with such estrogen receptor modulator fails to reach a predetermined clinical end point. In some embodiments, the predetermined clinical end point is a reduction in tumor size, a stabilization in tumor size, partial remission, complete remission or stable disease.

Some embodiments described herein provide a method of treating an aromatase inhibitor treatment-refractory metastatic breast cancer in a patient, comprising: (a) determining that the cancer is refractory to treatment with an aromatase inhibitor; and (b) provided that the cancer is refractory to treatment with an aromatase inhibitor, administering to the patient a therapeutically effective amount of an extract of Scutellaria Barbata D. Don. In some embodiments, the method further comprises determining that the cancer expresses nuclear estrogen receptor (ER) above a predetermined threshold. In some embodiments, the aromatase inhibitor is arimidex, aromasin or letrozole. In some embodiments, the method further comprises determining that the cancer is refractory to treatment with an aromatase inhibitor comprises administering the aromatase inhibitor to a patient and determining that treatment with such aromatase inhibitor fails to reach a predetermined clinical end point. In some embodiments, the predetermined clinical end point is a reduction in tumor size, a stabilization in tumor size, partial remission, complete remission or stable disease.

Some embodiments described herein provide a method of treating early stage breast cancer in a patient, comprising: In some embodiments, the method comprises treating breast cancer with one or more of surgery, radiation or chemotherapy; and administering to the patient a therapeutically effective amount of an extract of Scutellaria Barbata D. Don, wherein the therapeutically effective amount of the extract of Scutellaria Barbata D. Don is sufficient to prevent or reduce the likelihood that the cancer will recur. In some embodiments, the method further comprises determining that the cancer expresses nuclear estrogen receptor at a level below a predetermined threshold.

Some embodiments described herein provide a method of treating early stage breast cancer in a patient, comprising: (a) determining that the breast cancer expresses a nuclear estrogen receptor (ER); (b) providing that the breast cancer expresses an ER, administering to the patient an estrogen receptor modulator; and (c) administering to the patient a therapeutically effective amount of an extract of Scutellaria Barbata D. Don. In some embodiments, the estrogen receptor modulator is tamoxifen or raloxifene.

Some embodiments described herein provide a method of treating early stage breast cancer in a patient, comprising: (a) administering to the patient an aromatase inhibitor; and (b) administering to the patient a therapeutically effective amount of an extract of Scutellaria Barbata D. Don. In some embodiments, the aromatase inhibitor is arimidex, aromasin or letrozole. In some embodiments, the method further comprises determining a level of expression of nuclear estrogen receptor (ER) in the cancer. In some embodiments, the level of expression of ER is at or above a predetermined threshold and administration of the aromatase inhibitor and the therapeutically effective amount of an extract of Scutellaria Barbata D. Don are conditioned upon finding that the expression of ER is at or above the predetermined threshold.

Some embodiments described herein provide a method of treating advanced breast cancer in a patient, comprising: (a) administering to the patient an antitumor antibiotic; and (b) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient an antitumor antibiotic; and (ii) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient an antitumor antibiotic; and (ii) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan.

Some embodiments described herein provide a method of treating advanced breast cancer in a patient, comprising: (a) administering to the patient a nitrogen mustard; and (b) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient a nitrogen mustard; and (ii) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient a nitrogen mustard; and (ii) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously.

Some embodiments described herein provide a method of treating advanced breast cancer in a patient, comprising: (a) administering to the patient a taxane; and (b) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient a taxane; and (ii) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient a taxane; and (ii) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously.

Some embodiments described herein provide a method of treating advanced breast cancer in a patient, comprising: (a) administering to the patient an antimetabolite; and (b) administering to the patient a therapeutically effective amount of an extract of Scutellaria Barbata D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient an antimetabolite; and (ii) administering to the patient a therapeutically effective amount of an extract of Scutellaria Barbata D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient an antimetabolite; and (ii) administering to the patient a therapeutically effective amount of an extract of Scutellaria Barbata D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate.

Some embodiments described herein provide a method of treating advanced breast cancer in a patient, comprising: (a) administering to the patient an anti-cancer monoclonal antibody; and (b) administering to the patient a therapeutically effective amount of an extract of Scutellaria Barbata D. Don. In some embodiments, the antibody is alemtuzumab, bevacizumab, cetuximab, gemtuzumab, panitumumab, rituximab, tositumomab or trastuzumab.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient an anti-cancer monoclonal antibody; and (ii) administering to the patient a therapeutically effective amount of an extract of Scutellaria Barbata D. Don. In some embodiments, the antibody is alemtuzumab, bevacizumab, cetuximab, gemtuzumab, panitumumab, rituximab, tositumomab or trastuzumab.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient an anti-cancer monoclonal antibody; and (ii) administering to the patient a therapeutically effective amount of an extract of Scutellaria Barbata D. Don. In some embodiments, the antibody is alemtuzumab, bevacizumab, cetuximab, gemtuzumab, panitumumab, rituximab, tositumomab or trastuzumab.

Some embodiments described herein provide a method of treating advanced breast cancer in a patient, comprising: (a) administering to the patient a tyrosine kinase inhibitor; and (b) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient a tyrosine kinase inhibitor; and (ii) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient a tyrosine kinase inhibitor; and (ii) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib.

It is considered that since an extract of Scutellaria barbata D. Don is active in the treatment of estrogen receptor negative (ER⁻) breast cancer, it may also be active in the treatment of other cancers that lack estrogen receptor. Accordingly, some embodiments provided herein provide a method of treating cancer having low or no expression of nuclear estrogen receptor (ER) in a patient, comprising: (a) determining that the expression of ER in the cancer is below a predetermined threshold; and (b) provided that the expression of ER is below the predetermined threshold, administering to the patient a therapeutically effective amount of an extract of Scutellaria Barbata D. Don. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating an aromatase inhibitor treatment-refractory metastatic cancer in a patient, comprising: (a) determining that the cancer is refractory to treatment with an aromatase inhibitor; and (b) provided that the cancer is refractory to treatment with an aromatase inhibitor, administering to the patient a therapeutically effective amount of an extract of Scutellaria Barbata D. Don. In some embodiments, the method further comprises determining that the cancer expresses nuclear estrogen receptor (ER) above a predetermined threshold. In some embodiments, the aromatase inhibitor is arimidex, aromasin or letrozole. In some embodiments, determining that the cancer is refractory to treatment with an aromatase inhibitor comprises administering the aromatase inhibitor to a patient and determining that treatment with such aromatase inhibitor fails to reach a predetermined clinical end point. In some embodiments, the predetermined clinical end point is a reduction in tumor size, a stabilization in tumor size, partial remission, complete remission or stable disease. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) treating the cancer with one or more of surgery, radiation or chemotherapy; and (b) administering to the patient a therapeutically effective amount of an extract of Scutellaria Barbata D. Don, wherein the therapeutically effective amount of the extract of Scutellaria Barbata D. Don is sufficient to prevent or reduce the likelihood that the cancer will recur. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient an antitumor antibiotic; and (b) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments set forth herein provide a method of treating cancer in a patient, comprising: (a) administering to the patient a nitrogen mustard; and (b) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient a taxane; and (b) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient an antimetabolite; and (b) administering to the patient a therapeutically effective amount of an extract of Scutellaria Barbata D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient an anti-cancer monoclonal antibody; and (b) administering to the patient a therapeutically effective amount of an extract of Scutellaria Barbata D. Don. In some embodiments, the antibody is alemtuzumab, bevacizumab, cetuximab, gemtuzumab, panitumumab, rituximab, tositumomab or trastuzumab. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient a tyrosine kinase inhibitor; and (b) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a kit for treatment of cancer, comprising a therapeutically effective amount of a first chemotherapeutic agent comprising an extract of Scutellaria Barbata D. Don and a therapeutically effective amount of a second chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor. In some embodiments, the second chemotherapeutic agent is: (a) an aromatase selected from arimidex, aromasin and letrozole; (b) an antitumor antibiotic selected from daunorubicin, doxorubicin, epirubicin, idarubicin, mitoxantrone, valrubicin, actinomycin, bleomycin, mitomycin and plicamycin; (c) a nitrogen mustard selected from chlorambucil, chlormethine, cyclophosphamide, ifosfamide and melphalan; (d) a taxane selected from paclitaxel and docetaxel; (e) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine; (f) an anti-cancer monoclonal antibody selected from alemtuzumab, bevacizumab, cetuximab, gemtuzumab, panitumumab, rituximab, tositumomab and trastuzumab; or (g) a tyrosine kinase inhibitor selected from dasatinib, erlotinib, gefitinib, imatinib, lapatinib, nilotinib, sorafenib and sunitinib. In some embodiments, the kit further comprises a third chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor. In some embodiments, the third chemotherapeutic agent is: (a) an aromatase selected from arimidex, aromasin and letrozole; (b) an antitumor antibiotic selected from daunorubicin, doxorubicin, epirubicin, idarubicin, mitoxantrone, valrubicin, actinomycin, bleomycin, mitomycin and plicamycin; (c) a nitrogen mustard selected from chlorambucil, chlormethine, cyclophosphamide, ifosfamide and melphalan; (d) a taxane selected from paclitaxel and docetaxel; (e) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine; (f) an anti-cancer monoclonal antibody selected from alemtuzumab, bevacizumab, cetuximab, gemtuzumab, panitumumab, rituximab, tositumomab and trastuzumab; or (g) a tyrosine kinase inhibitor selected from dasatinib, erlotinib, gefitinib, imatinib, lapatinib, nilotinib, sorafenib and sunitinib.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows dose-response curves showing the response of several solid cancer tumor cells to aqueous extract of the herb of this invention.

FIG. 2 shows dose-response curves showing the response of several breast solid cancer tumor cells to aqueous extract of the herb of the invention.

FIG. 3 shows dose-response curves comparing the response of breast solid cancer tumor cells and normal breast epithelium to aqueous extract of the herb of this invention.

FIG. 4 shows gel electrophoresis plate, which demonstrates that nuclear DNA disintegration occurs during apoptosis of solid tumor cancer cells in contact with aqueous extracts of the herb of this invention.

FIG. 5 shows the effect of the herb extract of the invention administered intraperitoneally (IP) on the tumors of mice in a xenograft model.

FIG. 6 shows the effect of the herb extract administered by oral gavages and in interaction with cyclophosphamide administered in low dose in the drinking water on the tumors of mice in a xenograft model.

FIG. 7 shows that the herb extract induces apoptosis without activating caspases.

FIG. 8 shows that the herb extract in cell cycle analysis arrests the cells at the G1 phase.

FIG. 9 shows that illustrates that BZL101 leads to oxidative DNA damage. Formation of 8-oxoguanine, the most ubiquitous marker of DNA oxidation, was quantified through flow cytometric analysis of fixed permeabilzed cells incubated with avidin fluorescein, that was shown to bind relatively specifically to 8-oxoguanine. There is a clear increase in binding of avidin to BZL101 treated SKBr3 cells versus untreated cells.

FIG. 10 shows that the conversion of non-fluorescent CM-H₂DCFDA into fluorescent compound is indeed due to ROS. Incubation of cells with ROS scavenger N-acetyl-cysteine (NAC) prior to addition of BZL101 prevented most of the increase in ROS generation.

### DETAILED DESCRIPTION OF THE INVENTION

Some embodiments described herein provide a method of treating metastatic breast cancer having low or no expression of nuclear estrogen receptor (ER) in a patient, comprising: (a) determining that the expression of ER in the cancer is below a predetermined threshold; and (b) provided that the expression of ER is below the predetermined threshold, administering to the patient a therapeutically effective amount of an extract of Scutellaria Barbata D. Don.

Some embodiments described herein provide a method of treating an estrogen receptor modulator treatment-refractory metastatic breast cancer, comprising: (a) determining that the tumor is refractory to treatment with an estrogen receptor modulator; and (b) provided that the tumor is refractory to treatment with an estrogen receptor modulator, administering to the patient a therapeutically effective amount of an extract of Scutellaria Barbata D. Don. In some embodiments, the method further comprises determining that the tumor expresses nuclear estrogen receptor (ER) above a predetermined threshold. In some embodiments, the estrogen receptor modulator is tamoxifen or raloxifene. In some embodiments, the method further comprises determining that the tumor is refractory to treatment with an estrogen receptor modulator comprises administering the estrogen receptor modulator to a patient and determining that treatment with such estrogen receptor modulator fails to reach a predetermined clinical end point. In some embodiments, the predetermined clinical end point is a reduction in tumor size, a stabilization in tumor size, partial remission, complete remission or stable disease.

Some embodiments described herein provide a method of treating an aromatase inhibitor treatment-refractory metastatic breast cancer in a patient, comprising: (a) determining that the cancer is refractory to treatment with an aromatase inhibitor; and (b) provided that the cancer is refractory to treatment with an aromatase inhibitor, administering to the patient a therapeutically effective amount of an extract of Scutellaria Barbata D. Don. In some embodiments, the method further comprises determining that the cancer expresses nuclear estrogen receptor (ER) above a predetermined threshold. In some embodiments, the aromatase inhibitor is arimidex, aromasin or letrozole. In some embodiments, the method further comprises determining that the cancer is refractory to treatment with an aromatase inhibitor comprises administering the aromatase inhibitor to a patient and determining that treatment with such aromatase inhibitor fails to reach a predetermined clinical end point. In some embodiments, the predetermined clinical end point is a reduction in tumor size, a stabilization in tumor size, partial remission, complete remission or stable disease.

Some embodiments described herein provide a method of treating early stage breast cancer in a patient, comprising: In some embodiments, the method comprises treating breast cancer with one or more of surgery, radiation or chemotherapy; and administering to the patient a therapeutically effective amount of an extract of Scutellaria Barbata D. Don, wherein the therapeutically effective amount of the extract of Scutellaria Barbata D. Don is sufficient to prevent or reduce the likelihood that the cancer will recur. In some embodiments, the method further comprises determining that the cancer expresses nuclear estrogen receptor at a level below a predetermined threshold.

Some embodiments described herein provide a method of treating early stage breast cancer in a patient, comprising: (a) determining that the breast cancer expresses a nuclear estrogen receptor (ER); (b) providing that the breast cancer expresses an ER, administering to the patient an estrogen receptor modulator; and (c) administering to the patient a therapeutically effective amount of an extract of Scutellaria Barbata D. Don. In some embodiments, the estrogen receptor modulator is tamoxifen or raloxifene.

Some embodiments described herein provide a method of treating early stage breast cancer in a patient, comprising: (a) administering to the patient an aromatase inhibitor; and (b) administering to the patient a therapeutically effective amount of an extract of Scutellaria Barbata D. Don. In some embodiments, the aromatase inhibitor is arimidex, aromasin or letrozole. In some embodiments, the method further comprises determining a level of expression of nuclear estrogen receptor (ER) in the cancer. In some embodiments, the level of expression of ER is at or above a predetermined threshold and administration of the aromatase inhibitor and the therapeutically effective amount of an extract of Scutellaria Barbata D. Don are conditioned upon finding that the expression of ER is at or above the predetermined threshold.

Some embodiments described herein provide a method of treating advanced breast cancer in a patient, comprising: (a) administering to the patient an antitumor antibiotic; and (b) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient an antitumor antibiotic; and (ii) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient an antitumor antibiotic; and (ii) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan.

Some embodiments described herein provide a method of treating advanced breast cancer in a patient, comprising: (a) administering to the patient a nitrogen mustard; and (b) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient a nitrogen mustard; and (ii) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient a nitrogen mustard; and (ii) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously.

Some embodiments described herein provide a method of treating advanced breast cancer in a patient, comprising: (a) administering to the patient a taxane; and (b) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient a taxane; and (ii) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient a taxane; and (ii) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously.

Some embodiments described herein provide a method of treating advanced breast cancer in a patient, comprising: (a) administering to the patient an antimetabolite; and (b) administering to the patient a therapeutically effective amount of an extract of Scutellaria Barbata D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient an antimetabolite; and (ii) administering to the patient a therapeutically effective amount of an extract of Scutellaria Barbara D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient an antimetabolite; and (ii) administering to the patient a therapeutically effective amount of an extract of Scutellaria Barbata D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate.

Some embodiments described herein provide a method of treating advanced breast cancer in a patient, comprising: (a) administering to the patient an anti-cancer monoclonal antibody; and (b) administering to the patient a therapeutically effective amount of an extract of Scutellaria Barbata D. Don. In some embodiments, the antibody is alemtuzumab, bevacizumab, cetuximab, gemtuzumab, panitumumab, rituximab, tositumomab or trastuzumab.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient an anti-cancer monoclonal antibody; and (ii) administering to the patient a therapeutically effective amount of an extract of Scutellaria Barbara D. Don. In some embodiments, the antibody is alemtuzumab, bevacizumab, cetuximab, gemtuzumab, panitumumab, rituximab, tositumomab or trastuzumab.

Some embodiments described herein provide a method of treating advanced breast cancer in a patient, comprising: (a) determining an expression level of nuclear estrogen receptor (ER) in the cancer; (b) and, provided that the ER level determined in (a) exceeds a predetermined threshold: (i) administering to the patient an anti-cancer monoclonal antibody; and (ii) administering to the patient a therapeutically effective amount of an extract of Scutellaria Barbata D. Don. In some embodiments, the antibody is alemtuzumab, bevacizumab, cetuximab, gemtuzumab, panitumumab, rituximab, tositumomab or trastuzumab.

Some embodiments described herein provide a method of treating advanced breast cancer in a patient, comprising: (a) administering to the patient a tyrosinee kinase inhibitor; and (b) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib.

Some embodiments described herein provide a method of treating advanced breast cancer in a patient, comprising: (a) determining an expression level of nuclear estrogen receptor (ER) in the cancer; (b) and, provided that the ER level determined in (a) does not exceed a predetermined threshold: (i) administering to the patient a tyrosine kinase inhibitor; and (ii) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib.

Some embodiments described herein provide a method of treating advanced breast cancer in a patient comprising: (a) determining an expression level of nuclear estrogen receptor (ER) in the cancer; (b) and, provided that the ER level determined in (a) exceeds a predetermined threshold: (i) administering to the patient a tyrosine kinase inhibitor; and (ii) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib.

It is considered that since an extract of Scutellaria barbata D. Don is active in the treatment of estrogen receptor negative (ER⁻) breast cancer, it may also be active in the treatment of other cancers that lack estrogen receptor. Accordingly, some embodiments provided herein provide a method of treating cancer having low or no expression of nuclear estrogen receptor (ER) in a patient, comprising: (a) determining that the expression of ER in the cancer is below a predetermined threshold; and (b) provided that the expression of ER is below the predetermined threshold, administering to the patient a therapeutically effective amount of an extract of Scutellaria Barbata D. Don. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating an aromatase inhibitor treatment-refractory metastatic cancer in a patient, comprising: (a) determining that the cancer is refractory to treatment with an aromatase inhibitor; and (b) provided that the cancer is refractory to treatment with an aromatase inhibitor, administering to the patient a therapeutically effective amount of an extract of Scutellaria Barbata D. Don. In some embodiments, the method further comprises determining that the cancer expresses nuclear estrogen receptor (ER) above a predetermined threshold. In some embodiments, the aromatase inhibitor is arimidex, aromasin or letrozole. In some embodiments, determining that the cancer is refractory to treatment with an aromatase inhibitor comprises administering the aromatase inhibitor to a patient and determining that treatment with such aromatase inhibitor fails to reach a predetermined clinical end point. In some embodiments, the predetermined clinical end point is a reduction in tumor size, a stabilization in tumor size, partial remission, complete remission or stable disease. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) treating the cancer with one or more of surgery, radiation or chemotherapy; and (b) administering to the patient a therapeutically effective amount of an extract of Scutellaria Barbata D. Don, wherein the therapeutically effective amount of the extract of Scutellaria Barbata D. Don is sufficient to prevent or reduce the likelihood that the cancer will recur. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient an antitumor antibiotic; and (b) administering to the patient a therapeutically effective amount of an extract of Scutellaria Barbata D. Don. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the method further comprises administering to the patient a nitrogen mustard. In some embodiments, the nitrogen mustard is chlorambucil, chlormethine, cyclophosphamide, ifosfamide or melphalan. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments set forth herein provide a method of treating cancer in a patient, comprising: (a) administering to the patient a nitrogen mustard; and (b) administering to the patient a therapeutically effective amount of an extract of Scutellaria Barbata D. Don. In some embodiments, the nitrogen mustard is chlorambucil, chlormethine, cyclophosphamide, ifosfamide or melphalan. In some embodiments, the method further comprises administering to the patient an antitumor antibiotic. In some embodiments, the antitumor antibiotic is daunorubicin, doxorubicin, epirubicin, idarubicin, mitoxantrone, valrubicin, actinomycin, bleomycin, mitomycin or plicamycin. In some embodiments, the nitrogen mustard and the extract are administered sequentially. In some embodiments, the nitrogen mustard and the extract are administered simultaneously. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient a taxane; and (b) administering to the patient a therapeutically effective amount of an extract of Scutellaria Barbata D. Don. In some embodiments, the taxane is paclitaxel or docetaxel. In some embodiments, the taxane and the extract are administered sequentially. In some embodiments, the taxane and the extract are administered simultaneously. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient, comprising: (a) administering to the patient an antimetabolite; and (b) administering to the patient a therapeutically effective amount of an extract of Scutellaria Barbata D. Don. In some embodiments, the antimetabolite is a folate anticancer agent, a purine anticancer agent or a pyrimidine anticancer agent. In some embodiments, the antimetabolite is aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine or gemcitabine. In some embodiments, the extract is administered to the patient along with cyclophosphamide, 5FU and methotrexate. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient comprising: (a) administering to the patient an anti-cancer monoclonal antibody; and (b) administering to the patient a therapeutically effective amount of an extract of Scutellaria Barbata D. Don. In some embodiments, the antibody is alemtuzumab, bevacizumab, cetuximab, gemtuzumab, panitumumab, rituximab, tositumomab or trastuzumab. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a method of treating cancer in a patient comprising: (a) administering to the patient a tyrosine kinase inhibitor; and (b) administering to the patient a therapeutically effective amount of an extract of Scutellaria Barbata D. Don. In some embodiments, the tyrosine kinase inhibitor is dasatinib, erlotinib, gefitinib, imatinib, lapatinib, nilotinib, sorafenib or sunitinib. In some embodiments, the treated cancer may be selected from the group consisting of solid tumors, such as sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Some embodiments described herein provide a kit for treatment of cancer, comprising a therapeutically effective amount of a first chemotherapeutic agent comprising an extract of Scutellaria Barbata D. Don and a therapeutically effective amount of a second chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor. In some embodiments, the second chemotherapeutic agent is: (a) an aromatase selected from arimidex, aromasin and letrozole; (b) an antitumor antibiotic selected from daunorubicin, doxorubicin, epirubicin, idarubicin, mitoxantrone, valrubicin, actinomycin, bleomycin, mitomycin and plicamycin; (c) a nitrogen mustard selected from chlorambucil, chlormethine, cyclophosphamide, ifosfamide and melphalan; (d) a taxane selected from paclitaxel and docetaxel; (e) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine; (f) an anti-cancer monoclonal antibody selected from alemtuzumab, bevacizumab, cetuximab, gemtuzumab, panitumumab, rituximab, tositumomab and trastuzumab; or (g) a tyrosine kinase inhibitor selected from dasatinib, erlotinib, gefitinib, imatinib, lapatinib, nilotinib, sorafenib and sunitinib. In some embodiments, the kit further comprises a third chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor. In some embodiments, the third chemotherapeutic agent is: (a) an aromatase selected from arimidex, aromasin and letrozole; (b) an antitumor antibiotic selected from daunorubicin, doxorubicin, epirubicin, idarubicin, mitoxantrone, valrubicin, actinomycin, bleomycin, mitomycin and plicamycin; (c) a nitrogen mustard selected from chlorambucil, chlormethine, cyclophosphamide, ifosfamide and melphalan; (d) a taxane selected from paclitaxel and docetaxel; (e) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine; (f) an anti-cancer monoclonal antibody selected from alemtuzumab, bevacizumab, cetuximab, gemtuzumab, panitumumab, rituximab, tositumomab and trastuzumab; or (g) a tyrosine kinase inhibitor selected from dasatinib, erlotinib, gefitinib, imatinib, lapatinib, nilotinib, sorafenib and sunitinib.

As used herein, the indefinite article “a” or “an” is to be interpreted as meaning “at least one” unless further qualified. The conjunction “or” is, unless otherwise qualified, intended to be inclusive.

### Scutellaria Barbata Extract and Other Chemotherapeutic Agents

In some embodiments, the composition comprising an extract of Scutellaria Barbata can be administered in conjunction with one or more additional chemotherapeutic agents. The composition comprising an extract of Scutellaria Barbata can be combined in a single dosage form, or may be administered separately from one or more additional chemotherapeutic agents. Because Scutellaria Barbata extract is apparently highly orally available, a currently preferred method of co-administering a Scutellaria Barbata extract along with an additional chemotherapeutic agent is for each active agent to be administered in a separate dosage form. Where two or more chemotherapeutic agents aside from Scutellaria Barbata extract are administered, they may be combined in a single dosage form—e.g. a single injectable dosage form—or may be administered separately—e.g. in separate intravenous formulations. In some embodiments, Scutellaria Barbata extract may be administered along with the additional chemotherapeutic agent as part of a single chemotherapeutic schema. In other embodiments, Scutellaria Barbata extract may be administered as an adjuvant to prevent recurrence of cancer. In some embodiments, Scutellaria Barbata extract may be administered prior to administration of another chemotherapeutic agent or other chemotherapeutic agents in order to pre-sensitize cancerous cells to the other chemotherapeutic agent or agents. In some embodiments Scutellaria Barbata may be administered after other chemotherapeutic agents have been administered and the prior treatment has failed to achieve a particular predetermined end point, such as remission (partial or total), stable disease, etc.

A chemotherapeutically effective amount of a composition comprising Scutellaria Barbata extract may administered to a patient in the same therapeutic schema as one or more additionally therapeutic approaches, such as surgery, radiation and treatment with one or more chemotherapeutic agents. Chemotherapeutic agents may include alkylating agents, antimetabolites, spindle poison or mitotic inhibitor, cytotoxic antibiotic, topoisomerase inhibitor, monoclonal antibodies, photosensitizers, tyrosine kinase inhibitors, or other chemotherapeutic agents.

A chemotherapeutically effective amount of a composition comprising Scutellaria Barbata extract may be administered to a patient as an adjunct to prevent, or reduce the probability of, recurrence of cancer. The Scutellaria Barbata extract may be administered after cessation of, or hiatus from, one or more standard therapies, including surgery, radiation, treatment with one or more chemotherapeutic agents, or other adjunctive therapy. Chemotherapeutic agents may include alkylating agents, antimetabolites, spindle poison or mitotic inhibitor, cytotoxic antibiotic, topoisomerase inhibitor, monoclonal antibodies, photosensitizers, tyrosine kinase inhibitors, or other chemotherapeutic agents.

A chemotherapeutically effective amount of a composition comprising Scutellaria Barbata extract may be administered to a patient as a pre-treatment prior to administration of one or more other anti-cancer therapies, such as surgery, radiation or chemotherapy. Thus, a chemotherapeutically effective amount of Scutellaria Barbata extract may be administered to a patient as a pre-treatment prior to administration of one or more alkylating agents, antimetabolites, spindle poison or mitotic inhibitor, cytotoxic antibiotic, topoisomerase inhibitor, monoclonal antibodies, photosensitizers, tyrosine kinase inhibitors, or other chemotherapeutic agents.

Alkylating Agents

A therapeutically effective amount of Scutellaria Barbata extract may be administered as part of the same therapeutic schema as one or more alkylating agents (and optionally one or more additional chemotherapeutic agents, such as a topoisomerase inhibitor, an antimetabolite or a platinum complex). The therapeutically effective amount of Scutellaria Barbata extract may be administered in the same dosage form as the alkylating agent or in a different dosage form. In some currently preferred embodiments, the alkylating agent is administered intravenously and the therapeutically effective amount of Scutellaria Barbata extract is administered orally. The alkylating agent and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the alkylating agent. Alkylating agents that may be administered to a patient as part of a therapeutic schema including administration of a therapeutically effective amount of Scutellaria Barbata extract include nitrogen mustards, nitrosoureas, platinum complexes, busulfan, dacarbazine, procarbazine, temozolomide, thioTEPA and uramustine. Nitrogen mustards include chlorambucil, chlormethine, cyclophosphamide, ifosfamide, and melphalan. Nitrosoureas include carmustine, fotemustine, lomustine and streptozocin. Platinum complexes include carboplatin, cisplatin, oxaliplatin and BBR3464.

A therapeutically effective amount of Scutellaria Barbata extract may also be administered to a patient prior to administration of one or more alkylating agents (administered alone or in combination with one or more additional chemotherapeutic agents and/or as part of a therapeutic schema including radiation and/or surgery) in order to pre-sensitize the cancer to the alkylating agent(s). Alkylating agents that may be administered to a patient following pre-administration of a therapeutically effective amount of Scutellaria Barbata extract include nitrogen mustards, nitrosoureas, platinum complexes, busulfan, dacarbazine, procarbazine, temozolomide, thioTEPA and uramustine. Nitrogen mustards include chlorambucil, chlormethine, cyclophosphamide, ifosfamide, and melphalan. Nitrosoureas include carmustine, fotemustine, lomustine and streptozocin. Platinum complexes include carboplatin, cisplatin, oxaliplatin and BBR3464.

A therapeutically effective amount of Scutellaria Barbata extract may be administered as an adjunctive therapy to prevent or reduce the likelihood of recurrence of cancer after treatment with one or more alkylating agents (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Alkylating agents that may be administered to a patient prior to administration of a therapeutically effective amount of Scutellaria Barbata extract include nitrogen mustards, nitrosoureas, platinum complexes, busulfan, dacarbazine, procarbazine, temozolomide, thioTEPA and uramustine. Nitrogen mustards include chlorambucil, chlormethine, cyclophosphamide, ifosfamide, and melphalan. Nitrosoureas include carmustine, fotemustine, lomustine and streptozocin. Platinum complexes include carboplatin, cisplatin, oxaliplatin and BBR3464.

Antimetabolites

A therapeutically effective amount of Scutellaria Barbata extract may be administered as part of the same therapeutic schema as one or more antimetabolites (and optionally one or more additional chemotherapeutic agents, such as a topoisomerase inhibitor or a platinum complex). The therapeutically effective amount of Scutellaria Barbata extract may be administered in the same dosage form as the antimetabolite or in a different dosage form. In some currently preferred embodiments, the antimetabolite is administered intravenously and the therapeutically effective amount of Scutellaria Barbata extract is administered orally. The antimetabolite and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the antimetabolite. Antimetabolites that may be administered to a patient as part of a therapeutic schema including administration of a therapeutically effective amount of Scutellaria Barbata extract include folic acid derivatives, purine derivatives, and pyrimidine derivatives. Folic acid derivatives include aminopterin, methotrexate, pemetrexed and raltitrexed. Purine anticancer derivatives include cladribine, clofarabine, fludarabine, mercaptopurine, pentostatin and thioguanine. Pyrimidine anticancer derivatives include capecitabine, cytarabine, 5-fluorouracil (5FU), floxuridine and gemcitabine.

A therapeutically effective amount of Scutellaria Barbata extract may also be administered to a patient prior to administration of one or more antimetabolites (administered alone or in combination with one or more additional chemotherapeutic agents and/or as part of a therapeutic schema including radiation and/or surgery) in order to pre-sensitize the cancer to the antimetabolites. Antimetabolites that may be administered to a patient after administration of a therapeutically effective amount of Scutellaria Barbata extract include folic acid derivatives, purine derivatives, and pyrimidine derivatives. Folic acid derivatives include aminopterin, methotrexate, pemetrexed and raltitrexed. Purine anticancer derivatives include cladribine, clofarabine, fludarabine, mercaptopurine, pentostatin and thioguanine. Pyrimidine anticancer derivatives include capecitabine, cytarabine, 5-fluorouracil (5FU), floxuridine and gemcitabine.

A therapeutically effective amount of Scutellaria Barbata extract may be administered as an adjunctive therapy to prevent or reduce the likelihood of recurrence of cancer after treatment with one or more alkylating agents (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Antimetabolites that may be administered to a patient prior to administration of a therapeutically effective amount of Scutellaria Barbata extract include folic acid derivatives, purine derivatives, and pyrimidine derivatives. Folic acid derivatives include aminopterin, methotrexate, pemetrexed and raltitrexed. Purine anticancer derivatives include cladribine, clofarabine, fludarabine, mercaptopurine, pentostatin and thioguanine. Pyrimidine anticancer derivatives include capecitabine, cytarabine, 5-fluorouracil (5FU), floxuridine and gemcitabine.

Spindle Poisons/Mitotic Inhibitors

A therapeutically effective amount of Scutellaria Barbata extract may be administered as part of the same therapeutic schema as one or more mitotic inhibitors (and optionally one or more additional chemotherapeutic agents, such as a topoisomerase inhibitor, an antimetabolite or a platinum complex). The therapeutically effective amount of Scutellaria Barbata extract may be administered in the same dosage form as the mitotic inhibitor or in a different dosage form. In some currently preferred embodiments, the mitotic inhibitor is administered intravenously and the therapeutically effective amount of Scutellaria Barbata extract is administered orally. The mitotic inhibitor and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the mitotic inhibitor. Spindle poison/mitotic inhibitors that may be administered to a patient as part of a therapeutic schema including administration of a therapeutically effective amount of Scutellaria Barbata extract include taxanes and vinca alkaloids. Taxanes include paclitaxel and docetaxel. Vinca alkaloids include vinblastine, vincristine, vindesine and vinorelbine.

A therapeutically effective amount of Scutellaria Barbata extract may also be administered to a patient prior to administration of one or more mitotic inhibitors (administered alone or in combination with one or more additional chemotherapeutic agents and/or as part of a therapeutic schema including radiation and/or surgery) in order to pre-sensitize the cancer to the mitotic inhibitor(s). Spindle poison/mitotic inhibitors that may be administered to a patient after administration of a therapeutically effective amount of Scutellaria Barbata extract include taxanes and vinca alkaloids. Taxanes include paclitaxel and docetaxel. Vinca alkaloids include vinblastine, vincristine, vindesine and vinorelbine.

A therapeutically effective amount of Scutellaria Barbata extract may be administered as an adjunctive therapy to prevent or reduce the likelihood of recurrence of cancer after treatment with one or more mitotic inhibitors (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Spindle poison/mitotic inhibitors that may be administered to a patient prior to administration of a therapeutically effective amount of Scutellaria Barbata extract as an adjunct include taxanes and vinca alkaloids. Taxanes include paclitaxel and docetaxel. Vinca alkaloids include vinblastine, vincristine, vindesine and vinorelbine.

Cytotoxic/Antitumor Antibiotics

A therapeutically effective amount of Scutellaria Barbata extract may be administered as part of the same therapeutic schema as one or more cytotoxic antibiotics (and optionally one or more additional chemotherapeutic agents, such as a topoisomerase inhibitor, an antimetabolite or a platinum complex). The therapeutically effective amount of Scutellaria Barbata extract may be administered in the same dosage form as the cytotoxic antibiotic or in a different dosage form. In some currently preferred embodiments, the cytotoxic antibiotic is administered intravenously and the therapeutically effective amount of Scutellaria Barbata extract is administered orally. The cytotoxic antibiotic and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the cytotoxic antibiotic. Cytotoxic/antitumor antibiotics that may be administered to a patient as part of a therapeutic schema including administration of a therapeutically effective amount of Scutellaria Barbata extract include members of the anthracycline family, members of the streptomyces family and hydroxyurea. Antitumor antibiotics of the anthracycline family include daunorubicin, doxorubicin, epirubicin, idarubicin, mitoxantrone and valrubicin. Anticancer antibiotics of the streptomyces family of anticancer agents include actinomycin, bleomycin, mitomycin and plicamycin.

A therapeutically effective amount of Scutellaria Barbata extract may also be administered to a patient prior to administration of one or more antibiotics (administered alone or in combination with one or more additional chemotherapeutic agents and/or as part of a therapeutic schema including radiation and/or surgery) in order to pre-sensitize the cancer to the antitumor antibiotic(s). Cytotoxic/antitumor antibiotics that may be administered after administration of a therapeutically effective amount of Scutellaria Barbata extract include members of the anthracycline family, members of the streptomyces family and hydroxyurea. Antitumor antibiotics of the anthracycline family include daunorubicin, doxorubicin, epirubicin, idarubicin, mitoxantrone and valrubicin. Anticancer antibiotics of the streptomyces family of anticancer agents include actinomycin, bleomycin, mitomycin and plicamycin.

A therapeutically effective amount of Scutellaria Barbata extract may be administered as an adjunctive therapy to prevent or reduce the likelihood of recurrence of cancer after treatment with one or more antitumor antibiotics (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Cytotoxic/antitumor antibiotics that may be administered prior to administration of a therapeutically effective amount of Scutellaria Barbata extract include members of the anthracycline family, members of the streptomyces family and hydroxyurea. Antitumor antibiotics of the anthracycline family include daunorubicin, doxorubicin, epirubicin, idarubicin, mitoxantrone and valrubicin. Anticancer antibiotics of the streptomyces family of anticancer agents include actinomycin, bleomycin, mitomycin and plicamycin.

Topoisomerase Inhibitors

A therapeutically effective amount of Scutellaria Barbata extract may be administered as part of the same therapeutic schema as one or more topoisomerase inhibitors (and optionally one or more additional chemotherapeutic agents, such as an antimetabolite or a platinum complex). The therapeutically effective amount of Scutellaria Barbata extract may be administered in the same dosage form as the topoisomerase inhibitor or in a different dosage form. In some currently preferred embodiments, the topoisomerase inhibitor is administered intravenously and the therapeutically effective amount of Scutellaria Barbata extract is administered orally. The topoisomerase inhibitor and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the topoisomerase inhibitor. Topoisomerase inhibitors that may be administered to a patient as part of a therapeutic schema including administration of a therapeutically effective amount of Scutellaria Barbata extract include camptotheca and podophyllum. Camtotheca include camptothecin, topotecan and irinotecan; members of the group of podophyllum include etoposide and teniposide.

A therapeutically effective amount of Scutellaria Barbata extract may also be administered to a patient prior to administration of one or more topoisomerase inhibitors (administered alone or in combination with one or more additional chemotherapeutic agents and/or as part of a therapeutic schema including radiation and/or surgery) in order to pre-sensitize the cancer to the topoisomerase inhibitor(s). Topoisomerase inhibitors that may be administered to a patient after administration of a therapeutically effective amount of Scutellaria Barbata extract include camptotheca and podophyllum. Camtotheca include camptothecin, topotecan and irinotecan; members of the group of podophyllum include etoposide and teniposide.

A therapeutically effective amount of Scutellaria Barbata extract may be administered as an adjunctive therapy to prevent or reduce the likelihood of recurrence of cancer after treatment with one or more topoisomerase inhibitors (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Topoisomerase inhibitors that may be administered to a patient prior to administration of a therapeutically effective amount of Scutellaria Barbata extract include camptotheca and podophyllum. Camtotheca include camptothecin, topotecan and irinotecan; members of the group of podophyllum include etoposide and teniposide.

CI (Anti-Cancer) Monoclonal Antibodies

A therapeutically effective amount of Scutellaria Barbata extract may be administered as part of the same therapeutic schema as one or more monoclonal antibodies that is effective in the treatment of cancer (and optionally one or more additional chemotherapeutic agents, such as a topoisomerase inhibitor, an antimetabolite or a platinum complex). The therapeutically effective amount of Scutellaria Barbata extract may be administered in the same dosage form as the monoclonal antibody or in a different dosage form. In some currently preferred embodiments, the monoclonal antibody is administered intravenously and the therapeutically effective amount of Scutellaria Barbata extract is administered orally. The monoclonal antibody and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the monoclonal antibody. Anti-cancer monoclonal antibodies that may be administered to a patient as part of a therapeutic schema including administration of a therapeutically effective amount of Scutellaria Barbata extract include: alemtuzumab, bevacizumab, cetuximab, gemtuzumab, panitumumab, rituximab, tositumomab and trastuzumab.

A therapeutically effective amount of Scutellaria Barbata extract may also be administered to a patient prior to administration of one or more monoclonal antibodies (administered alone or in combination with one or more additional chemotherapeutic agents and/or as part of a therapeutic schema including radiation and/or surgery) in order to pre-sensitize the cancer to the monoclonal antibody(ies). Anti-cancer monoclonal antibodies that may be administered to a patient after administration of a therapeutically effective amount of Scutellaria Barbata extract include: alemtuzumab, bevacizumab, cetuximab, gemtuzumab, panitumumab, rituximab, tositumomab and trastuzumab.

A therapeutically effective amount of Scutellaria Barbata extract may be administered as an adjunctive therapy to prevent or reduce the likelihood of recurrence of cancer after treatment with one or more monoclonal antibodies (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Anti-cancer monoclonal antibodies that may be administered to a patient prior to administration of a therapeutically effective amount of Scutellaria Barbata extract include: alemtuzumab, bevacizumab, cetuximab, gemtuzumab, panitumumab, rituximab, tositumomab and trastuzumab.

Photosensitizers

A therapeutically effective amount of Scutellaria Barbata extract may be administered as part of the same therapeutic schema as one or more photosensitizers (and optionally one or more additional chemotherapeutic agents, such as a topoisomerase inhibitor, an antimetabolite or a platinum complex). The therapeutically effective amount of Scutellaria Barbata extract may be administered in the same dosage form as the photosensitizer or in a different dosage form. In some currently preferred embodiments, the photosensitizer is administered intravenously and the therapeutically effective amount of Scutellaria Barbata extract is administered orally. The photosensitizer and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the photosensitizer. Photosensitizers that may be administered to a patient as part of a therapeutic schema including administration of a therapeutically effective amount of Scutellaria Barbata extract include aminolevulinic acid, methyl aminolevulinate, porfimer sodium and verteporfin.

A therapeutically effective amount of Scutellaria Barbata extract may also be administered to a patient prior to administration of one or more photosensitizers (administered alone or in combination with one or more additional chemotherapeutic agents and/or as part of a therapeutic schema including radiation and/or surgery) in order to pre-sensitize the cancer to the photosensitizer (s). Photosensitizers that may be administered after administration of a therapeutically effective amount of Scutellaria Barbata extract include aminolevulinic acid, methyl aminolevulinate, porfimer sodium and verteporfin.

A therapeutically effective amount of Scutellaria Barbata extract may be administered as an adjunctive therapy to prevent or reduce the likelihood of recurrence of cancer after treatment with one or more photosensitizers (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Photosensitizers that may be administered prior to administration of a therapeutically effective amount of Scutellaria Barbata extract include aminolevulinic acid, methyl aminolevulinate, porfimer sodium and verteporfin.

Tyrosine Kinase Inhibitors

A therapeutically effective amount of Scutellaria Barbata extract may be administered as part of the same therapeutic schema as one or more tyrosine kinase inhibitors (and optionally one or more additional chemotherapeutic agents, such as a topoisomerase inhibitor, an antimetabolite or a platinum complex). The therapeutically effective amount of Scutellaria Barbata extract may be administered in the same dosage form as the tyrosine kinase inhibitor or in a different dosage form. In some currently preferred embodiments, the tyrosine kinase inhibitor is administered intravenously and the therapeutically effective amount of Scutellaria Barbata extract is administered orally. The tyrosine kinase inhibitor and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the tyrosine kinase inhibitor. Tyrosine kinase inhibitors that may be administered to a patient as part of a therapeutic schema including administration of a therapeutically effective amount of Scutellaria Barbata extract include dasatinib, erlotinib, gefitinib, imatinib, lapatinib, nilotinib, sorafenib, sunitinib.

A therapeutically effective amount of Scutellaria Barbata extract may also be administered to a patient prior to administration of one or more tyrosine kinase inhibitors (administered alone or in combination with one or more additional chemotherapeutic agents and/or as part of a therapeutic schema including radiation and/or surgery) in order to pre-sensitize the cancer to the tyrosine kinase inhibitor(s). Tyrosine kinase inhibitors that may be administered to a patient after administration of a therapeutically effective amount of Scutellaria Barbata extract include dasatinib, erlotinib, gefitinib, imatinib, lapatinib, nilotinib, sorafenib, sunitinib.

A therapeutically effective amount of Scutellaria Barbata extract may be administered as an adjunctive therapy to prevent or reduce the likelihood of recurrence of cancer after treatment with one or more tyrosine kinase inhibitors (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Tyrosine kinase inhibitors that may be administered to a patient prior to administration of a therapeutically effective amount of Scutellaria Barbata extract include dasatinib, erlotinib, gefitinib, imatinib, lapatinib, nilotinib, sorafenib, sunitinib.

Other Anticancer Active Agents

A therapeutically effective amount of Scutellaria Barbata extract may be administered as part of the same therapeutic schema as one or more other anticancer agents (and optionally one or more additional chemotherapeutic agents, such as a topoisomerase inhibitor, an antimetabolite or a platinum complex). The therapeutically effective amount of Scutellaria Barbata extract may be administered in the same dosage form as the other anticancer agent or in a different dosage form. In some currently preferred embodiments, the other anticancer agent is administered intravenously and the therapeutically effective amount of Scutellaria Barbata extract is administered orally. The other anticancer agent and the therapeutically effective amount of Scutellaria Barbata extract may be administered on the same day or different days within a pre-determined treatment cycle, which may be 1 to 30 days in length (or more), and may be followed by another treatment cycle or a suitable wash-out period of predetermined length (e.g. from 1 to 60 days or more). In some embodiments, the administration of a therapeutically effective amount of Scutellaria Barbata extract may continue during a wash-out period for the other anticancer agent. Other anticancer active agents that may be administered to a patient as part of a therapeutic schema including administration of a therapeutically effective amount of Scutellaria Barbata extract include retinoids (such as alitretinoin and tretinoin), altretamine, amsacrine, anagrelide, arsenic trioxide, asparaginase (pegaspargase), bexarotene, bortezomib, denileukin diftitox, estramustine, masoprocol and mitotane.

A therapeutically effective amount of Scutellaria Barbata extract may also be administered to a patient prior to administration of one or more other anticancer agent (administered alone or in combination with one or more additional chemotherapeutic agents and/or as part of a therapeutic schema including radiation and/or surgery) in order to pre-sensitize the cancer to the other anticancer agent (s). Other anticancer active agents that may be administered to a patient after administration of a therapeutically effective amount of Scutellaria Barbata extract include retinoids (such as alitretinoin and tretinoin), altretamine, amsacrine, anagrelide, arsenic trioxide, asparaginase (pegaspargase), bexarotene, bortezomib, denileukin diftitox, estramustine, masoprocol and mitotane.

A therapeutically effective amount of Scutellaria Barbata extract may be administered as an adjunctive therapy to prevent or reduce the likelihood of recurrence of cancer after treatment with one or more other anticancer agents (administered alone or in combination with one or more additional chemotherapeutic agents and/or as part of a therapeutic schema including radiation and/or surgery). A therapeutically effective amount of Scutellaria Barbata may also be administered to a patient after one or more therapeutic approaches has failed to reach a predetermined end point. Other anticancer active agents that may be administered to a patient prior to administration of a therapeutically effective amount of Scutellaria Barbata extract include retinoids (such as alitretinoin and tretinoin), altretamine, amsacrine, anagrelide, arsenic trioxide, asparaginase (pegaspargase), bexarotene, bortezomib, denileukin diftitox, estramustine, masoprocol and mitotane.

Scutellaria Barbata D. Don (BZL)

Scutellaria barbata extract, when placed in contact with solid tumor cancer cells, inhibits the activity, that is the growth and/or proliferation, of the cells. The herb is selected from the species Scutellaria barbata D. Don of the Labiatae Family. Herba Scutellaria Barbata D. Don (Lamiaceae) of the Labiatae family—Ban Zhi Lian (BZL) is grown mainly in areas southeastern of the Yellow River (Huang Po) in the provinces of Sichuan, Jiangsu, Jiangxi, Fujian, Guangdong, Guangxi and Shaanxi but not exclusively. The plant is harvested in late summer and early autumn after it blooms (May-June). The aerial part is cut from the root. Only the aerial part (leaves and stems) is used for preparation of the extract of Scutellaria barbata D. Don, as described herein. The herb is dried in the sun and packed as a whole plant. The herb is received with no separation between leaves and stems.

Thus, except as otherwise specifically qualified herein, the term “extract” refers to an extract of the aerial portion (leaves and stems) of Scutellaria barbata D. Don. Except as otherwise specifically qualified herein, the term “herb” refers to the aerial portion of Scutellaria barbata D. Don. Except as otherwise specifically qualified herein the term “a pharmaceutically effective amount” of extract means an amount of extract sufficient to bring about a positive clinical outcome in at least one patient. A positive clinical outcome will be measured by conventional clinical standards know to the skilled oncologist. Some suitable positive clinical outcomes include partial remission, complete remission, a reduction in tumor size, stable tumor size, prevention of metastasis for a period exceeding at least about 3 months, at least about 6 months, at least about 9 months or at least about 12 months, extension of expected life expectancy, prevention of recurrence of a cancer, extension of the expected time necessary for recurrence of cancer. It is expected that an aspect of the invention will be that when the extract is administered in conjunction with another chemotherapeutic agent, the amount of extract that will be necessary to achieve a positive clinical outcome—and thus the pharmaceutically effective amount—will be less than that necessary when the extract is used as a single entity agent. A process of manufacturing a dose of extract is set forth in detail below. For purposes of this disclosure, the pharmaceutically effective amount of extract will be the dry solid portion of a hot water or ethanolic extract from approximately 1-20,000 g of Scutellaria barbata D. Don. In some embodiments, the pharmaceutically effective dose will be the dry solid portion of a hot aqueous or ethanolic extract of about 10 to about 2000 g of Scutellaria barbata D. Don.

As is described in the Detailed Description section, below, the herb is substantially more active in inhibiting the activity of different types of cancer cells. It is therefore a presently preferred aspect of this invention that the herbal extract obtained from the species Scutellaria barbata. It is a particularly presently preferred aspect of this invention that the herbal extract is obtained from Scutellaria barbata D. Don.

It has been previously shown that an extract of Scutellaria barbata D. Don. inhibits solid tumors in vitro. Some solid tumor cancer cell lines in which the extract is active include: SKBR3 cell, a MCF7 cell, a MDA-MB231 cell, a BT474 cell or a MCNeuA cell (breast cancer cells), A549 cell, LLC cell (Lung Cancer cells), Panc1 cells, Panc02 cells (Pancreatic cancer cells), PC-3 cells LNCaP cells (Prostate Cancer cells), OVCAR cells, SKOV3 cells (Ovarian Cancer cells).

Table 1 contains a description of the herb, from which extracts are obtained, listed by family, genus, species and traditional Chinese name.

TABLE 1

| Family | genus | Species | Chinese name | Herb part |
|---|---|---|---|---|
| Lamiaceae | Scutellaria | Barbata D. Don | Ban Zhi Lian | aerial |

Table 2A shows the degree of inhibition of the activity of several in vitro solid breast cancer tumor cell lines by the extract.

TABLE 2A

| MCF7 | SKBR3 | MDA-MB231 | BT474 | MCNeuA |
|---|---|---|---|---|
| ++ | ++ | ++ | + | ++ |

Table 2B shows the degree of inhibition of the activity of several in vitro solid cancer tumor cell lines by the extract.

TABLE 2B

| Lung Cancer | | Pancreatic Cancer | | Prostate Cancer | | Breast Cancer | | Breast Normal |
|---|---|---|---|---|---|---|---|---|
| A549 | LLC | Panel | Panc02 | PC-3 | LNCaP | MCF7 | MCNeuA | HuMEC |
| + | ++ | + | ++ | + | + | ++ | ++ | − |
| 1424 | 492 | 1054 | 594 | 1035 | 1516 | 818 | 619 | |

− <50% inhibition, + 51-75% inhibition, ++ >75% inhibition, IC₅₀ values (μg/ml)

The active ingredients in the extract are not known. The extract loses activity when reconstituted after drying, as well as when the extract is separated through physical and chemical means. The known chemical ingredients in the plant are scutellarin, scutelarein, carthamidin, isocarthamidin and wagonin.

An extract comprises residue of soluble solids obtained after the herb is for example, without limitation, chopped, crushed, pulverized, minced or otherwise treated to increase the effective surface area of the surface area of the herb and is placed in intimate contact with a liquid, usually, but not necessarily, under conditions of agitation and elevated temperature. Then, after a period of time under the foregoing conditions the mixture is filtered to remove a substantial portion of insoluble solids and the liquid is removed by, for example but not limitation, evaporation or freeze drying to produce the aforementioned residue. This residue contains soluble solids, which are believed to comprise the active agent in the extract, and in some cases optionally a portion of insoluble solids that were not removed by previous filtration. The liquid used to obtain an extract may be water or an organic solvent, for example, without limitation, an alcohol such as methyl, ethyl or isopropyl alcohol, a ketone such as acetone or methyl ethyl ketone (MEK), an ester such as ethyl acetate, an organochlorine compound such as methylene chloride, chloroform or carbon tetrachloride, a hydrocarbon such as pentane, hexane or benzene and the like. An extract may also be obtained by using a combination of these solvents with or without water.

As used herein, “administer”, “administering” or “administration” refers to the delivery of an extract or of a pharmaceutical composition containing an extract to a patient.

A “patient” refers to any higher organism that is susceptible to solid tumor cancers. Examples of such higher organisms include, without limitation, mice, rats, rabbits, dogs, cats, horses, cows, pigs, sheep, fish and reptiles. In currently preferred embodiments, the term “patient” refers to a human being.

As used herein, the term “therapeutically effective amount” refers to that amount of an extract or combination of extracts of this invention which has the effect of (1) reducing the size of the tumor; (2) inhibiting (that is, slowing to some extent, preferably stopping) tumor metastasis; (3) inhibiting to some extent (that is slowing to some extent, preferably stopping) tumor growth; and/or, (4) relieving to some extent (or preferably eliminating) one or more symptoms associated with cancer (5) stabilizing the growth of the tumor, (6) extending the time to disease progression, (7) improving overall survival.

As used herein, a “pharmaceutical composition” refers to a mixture of an extract described herein with another component or components, such as physiologically acceptable carriers and excipients. The purpose of a pharmacological composition is to facilitate administration of an extract or extracts of this invention to patient. In some currently preferred embodiments, the pharmaceutical composition can include water. In some currently preferred embodiments, the pharmaceutical composition can additionally include a flavor-masking agent.

As used herein, the term “pharmaceutically acceptable” means that the modified agent or excipient is generally regarded as acceptable for use in a pharmaceutical composition.

As used herein, a “physiologically acceptable carrier” refers to a carrier or diluent that does not cause significant irritation to an organism and does not abrogate the biological activity and properties of the administered composition.

As used herein, an “excipient” refers to an inert substance added to a pharmaceutical composition to further facilitate administration of an extract or extracts of this invention. Examples, without limitation, of excipients include calcium carbonate, calcium phosphate, various sugars and types of starch, cellulose derivatives, gelatin, vegetable oils and polyethylene glycols.

At one time, botanical agents were the most significant group of substances used by healers to treat patients. According to a WHO survey, 80% of the world's population still relies heavily on herbal medicine as their primary source of therapy. In Western culture one-quarter of the active components of currently prescribed drugs were first identified in plants and over half of the 50 most popular drugs today are derived from plant materials. In addition, over 60% of chemotherapeutic agents used in the treatment of cancer are derived from natural substances.

A useful strategy for the discovery of biologically active compounds from plants is the ethno-pharmacological approach which uses information about traditional medicinal uses of plants. The long history of a plant's use in treating a disorder, regardless of whether the disorder is well-characterized, e.g., skin rash, or is rather more nebulous, e.g., hot blood, is a clear indicator that something in the plant has some manner of beneficial effect on a disorder, otherwise the use of the plant would have faded in time. Furthermore, the fact that homeopathic practitioners have been administering the plant or an extract thereof to human patients for, often, centuries provides a compelling argument for the safety of the plant or its extracts in human beings.

Such alternative approaches to medicine are becoming more and more widely accepted and used in the United States as well to treat a broad spectrum of conditions as well as to maintain wellness. It is estimated that one in two Americans currently uses alternative therapies at one time or another. In particular, the most popular complementary or fully alternative approach to the treatment of their cancers by patients is botanical agents/herbal medicines.

Traditional Chinese medicine (TCM) is often the treatment modality of choice by cancer patients opting for an alternative approach to dealing with their ailment. Patients use TCM both as anti-cancer agents and to alleviate the side effects of standard chemotherapy. However, TCM lacks the scientifically sound methodology required of Western pharmacology and the use of TCM is often hit or miss in its effectiveness. There remains a need for the discovery of specific herbal extracts and combinations thereof that have a specific utility and for which there is scientific evidence as to why they work in that use. This invention provides such extract and compositions decoction.

### Pharmaceutical Compositions and Modes of Administrations

An extract of this invention can be administered to a patient either as a “tea,” without combination with any other substances or further manipulation, or it can be administered as a pharmaceutical composition where the extract is mixed with suitable carriers or recipient(s). In treating a patient exhibiting a disorder of interest, a therapeutically effective amount of the extract is administered. A therapeutically effective amount refers to that amount of the extract that results in amelioration of symptoms or a prolongation of survival in a patient, and may include destruction of a malignant tumor of a microbial infection.

When administered without combination with any other substances, the composition comprising extract of Scutellaria Barbata (especially Scutellaria Barbata D. Don) may be encased in a suitable capsule, such as a gelatin capsule. When administered in admixture with other excipients, adjuvants, binders, diluents, disintegrants, etc., the dry extract of Scutellaria Barbata may be compressed into a capsule or caplet in a conventional manner that is well-known in the art.

Toxicity and therapeutic efficacy of the extracts, i.e., determining the LD50 (the dose lethal to 50% of the population) and the ED50 (the dose therapeutically effective in 50% of the population) can be determined by standard pharmaceutical procedures in cell cultures or experimental animals. The dose ratio between toxic and therapeutic effects is the therapeutic index and it can be expressed as the ratio LD50/ED50. Extracts that exhibit large therapeutic indices are preferred. The data obtained from these cell culture assays and animal studies can be used in formulating a range of dosages for use in humans, in particular for internal use, that include ED50 with little or no toxicity. The dosage may vary within this range depending upon the dosage form employed and the route of administration utilized. In general, since the extracts used in the methods of this invention have been used in TCM, they are known to be relatively non-toxic to humans and therefore it is expected that they will exhibit large therapeutic indices.

For any extract used in the method of invention, the therapeutically effective dose can be estimated initially from cell culture assays. For example, a dose can be formulated in animal models to achieve a circulating plasma concentration range that includes the IC50 as determined in cell culture. Such information can be used to more accurately determine useful doses in humans. Levels in plasma may be measured, for example, by HPLC.

The exact formulation, route of administration and dosage can be chosen by the individual physician in view of the patient's condition and based on knowledge of TCM. (See e.g. Fingl et al., in The Pharmacological Basis of Therapeutics, 1975, Ch. 1, p. 1). It should be noted that the attending physician would know how and when to terminate, interrupt, or adjust administration due to toxicity, or organ dysfunction. Conversely, the attending physician would also know to adjust treatment to higher levels if the clinical response is not adequate. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

If desired, standard western medicine techniques for formulation and administration may be used, such as those found in Remington's Pharmaceutical Sciences, 18^{th} ed., Mack Publishing Co., Easton, Pa. (1990). Suitable routes may include: oral, rectal, transdermal, vaginal, transmucosal, or intestinal administration; parenteral delivery, including intramuscular, subcutaneous, intramedullary injections; as well as intrathecal, direct intraventricular, intravenous, intraperitoneal, intranasal, or intraocular injections, to name a just a few. In particular embodiments, the extract of the invention is administered orally.

For injection, an extract of this invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate an extract herein use in the methods disclosed for the practice of this invention in dosages suitable for systemic administration is within the scope of the invention. With proper choice of carrier and suitable manufacturing practice, an extract of the present invention, in particular those formulated as solutions, may be administered parenterally, such as by intravenous injection. Likewise, an extract can be formulated, using pharmaceutically acceptable carriers well known in the art, into dosages suitable for oral administration. Such carriers enable extracts to be formulated as tablets, pills, capsules, liquids, gels, syrups, slurries, suspensions and the like, for oral ingestion by a patient to be treated.

Pharmaceutical compositions suitable for use in the present invention are compositions wherein an extract is contained in an effective amount to achieve its intended purpose. Determination of the effective amount is well within the capability of those skilled in the art, especially in light of the detailed disclosure provided herein. A pharmaceutical composition may contain suitable pharmaceutically acceptable carriers including excipients and auxiliaries that facilitate processing of the extracts into preparations that can be used pharmaceutically. The preparations formulated for oral administration may be in the form of tablets, dragees, capsules, or solutions. The pharmaceutical compositions of the present invention may be manufactured in a manner that is itself known, e.g., by means of convention mixing, dissolving, granulating, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutically formulations for parenteral administration include aqueous solutions of an extract in water-soluble form. Additionally, suspensions of an extract may be prepared as appropriate oily injection suspensions may contain substances that increase the viscosity of the suspension, such as sodium carboxymethyl cellulose, sorbitol, or dextran. Optionally, the suspension may also contain suitable stabilizers or agents that increase the solubility of an extract to allow for the preparation of highly concentrated solutions.

Pharmaceutical preparations for oral use can be obtained by combining an extract with solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum Arabic, talc, polyvinyl pyrrolidone, carpool gel, polyethylene glycol, and/or titanium dioxide, lacquer solutions, and suitable organic solvents or solvent mixtures. Dyestuffs or pigments may be added to the tablets or dragee coatings for identification or to characterize different combinations of extracts and/or doses.

Pharmaceutical preparations that can be used orally include push-fit capsules made of gelatin, as well as soft, sealed capsules made of gelatin and a plasticizer, such as glycerol or sorbitol. The push-fit capsules contain the extract in admixture with fillers such as lactose, binders such as starches, and/or lubricants such as talc or magnesium separate and, optionally, stabilizers. In soft capsules, the extract may be dissolved or suspended in suitable liquids, such as fatty oils, liquid paraffin, or liquid polyethylene glycols.

The dosage of extract of Scutellaria barbata D. Don will vary depending upon the tumor type, the stage of disease, the species of patient and the individual patient. In some embodiments, the amount of extract of Scutellaria barbata D. Don (BZL) administered to a human patient will be the dry solid residue extracted from about 0.1 g to about 20,000 g of dried solid plant parts of BZL. In some embodiments, the effective dose is the dry solid residue extracted from about 1 to about 1000 g of BZL. In some embodiments, the effective dose will be the dry solid residue extracted from about 10 to about 800 g of BZL.

### Treatment of Cancers

Extracts of Scutellaria barbata D. Don may be used to treat solid tumors. Such tumors may include so-called estrogen receptor negative (ER⁻) breast cancer, estrogen receptor positive (ER⁺) cancer, and other solid tumor cancers. As used herein, the terms “estrogen receptor negative breast cancer” and “estrogen receptor positive breast cancers,” have meanings commonly ascribed to them in the art. The person skilled in the art will recognize that the terms “positive” and “negative” are relative terms describing levels of expression in a cell. In general, saying that a cell is “negative” for expression of a particular cell product means that the level of expression detected, if any, falls below a predetermined threshold. That threshold may be a detection limit, a background noise level or some arbitrary cutoff known and understood by one of skill in the art. As extracts of Scutellaria barbata D. Don do not necessarily require presence of ERα or ERβ in order to induce apoptosis in solid cancer cells, it is considered that doses of Scutellaria barbata D. Don may be used to treat, inter alia, either ER⁺ or ER⁻ breast cancers as well as other solid tumors. The dose of Scutellaria barbata D. Don extract may vary, however it is considered that a dose comprising the dry soluble portion of a hot water or ethanolic extract of about 1 to about 20,000 g, especially about 50 to about 10,000 g of dry aerial portions of Scutellaria barbata D. Don, is a therapeutically effective dose. When used in combination with another chemotherapeutic agents, the dose may be lowered to take advantage of synergetic effects. C that extracts of Scutellaria barbata D. Don may be used to treat include sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma.

Kits

Also provided herein are kits for treatment of cancer. In some embodiments, the kits comprise two or more active chemotherapeutic agents, at least one of which comprises an extract of Scutellaria barbata D. Don. In some embodiments, a first chemotherapeutic agent comprises an extract of Scutellaria barbata D. Don in an oral dosage form. In some embodiments, the second chemotherapeutic agent is in an oral or parenteral dosage form. Suitable parenteral dosage forms include intravenous or intraperitoneal injections. Kits can also contain instructions for administration of the extract of Scutellaria barbata D. Don and/or the second chemotherapeutic agent. In some embodiments, the kit will contain sufficient extract of Scutellaria barbata D. Don for administration over 1, 2, 3, 4 or more weeks. In some embodiments, the dosage of extract of Scutellaria barbata D. Don will be divided into daily or twice daily doses. The daily dose of extract of Scutellaria barbata D. Don may vary depending on the second chemotherapeutic agent, the disease to be treated, the condition of the patient, etc. In general, the daily dose of extract of Scutellaria barbata D. Don will be the dried soluble extract of about 1 to 20,000 g, 10 to 10,000 g or 50 to 5000 g of dried aerial portion of Scutellaria barbata D. Don. The daily dose may be divided into 2, 3, 4 or more doses per day. When administered as a tea, the doses may be combined with a flavor or flavor-masking agent in order to enhance palatability.

Some embodiments described herein provide a kit for treatment of cancer, comprising a therapeutically effective amount of a first chemotherapeutic agent comprising an extract of Scutellaria Barbata D. Don and a therapeutically effective amount of a second chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor. In some embodiments, the second chemotherapeutic agent is: (a) an aromatase selected from arimidex, aromasin and letrozole; (b) an antitumor antibiotic selected from daunorubicin, doxorubicin, epirubicin, idarubicin, mitoxantrone, valrubicin, actinomycin, bleomycin, mitomycin and plicamycin; (c) a nitrogen mustard selected from chlorambucil, chlormethine, cyclophosphamide, ifosfamide and melphalan; (d) a taxane selected from paclitaxel and docetaxel; (e) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine; (f) an anti-cancer monoclonal antibody selected from alemtuzumab, bevacizumab, cetuximab, gemtuzumab, panitumumab, rituximab, tositumomab and trastuzumab; or (g) a tyrosine kinase inhibitor selected from dasatinib, erlotinib, gefitinib, imatinib, lapatinib, nilotinib, sorafenib and sunitinib. In some embodiments, the kit further comprises a third chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor. In some embodiments, the third chemotherapeutic agent is: (a) an aromatase selected from arimidex, aromasin and letrozole; (b) an antitumor antibiotic selected from daunorubicin, doxorubicin, epirubicin, idarubicin, mitoxantrone, valrubicin, actinomycin, bleomycin, mitomycin and plicamycin; (c) a nitrogen mustard selected from chlorambucil, chlormethine, cyclophosphamide, ifosfamide and melphalan; (d) a taxane selected from paclitaxel and docetaxel; (e) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine; (f) an anti-cancer monoclonal antibody selected from alemtuzumab, bevacizumab, cetuximab, gemtuzumab, panitumumab, rituximab, tositumomab and trastuzumab; or (g) a tyrosine kinase inhibitor selected from dasatinib, erlotinib, gefitinib, imatinib, lapatinib, nilotinib, sorafenib and sunitinib.

### EXAMPLES

The herb from which the extracts of this invention were obtained were purchased from Shen Nong Herbs, Berkeley, Calif. Their identity was confirmed by reference to traditional pharmaceutical literature.

### Preparative Example 1

### Preparation of BZL101 for In Vitro and Mouse Experiments

Herbal extract was prepared as “boiled teas”, which is how most are prepared for use in traditional treatment regimes. Aqueous extracts were prepared by adding 7.5 g of dry ground herb to 125 ml distilled water, bringing the mixture to a boil and then simmering for 45 minutes. The mixture was cooled, during which period most of the solids sank to the bottom of the vessel. The aqueous layer was carefully decanted off of the residual solids, centrifuged for 5 minutes at 1500 rpm, sterile filtered through a 0.45 μm filter and stored at 4° C. until used. Generally, the extracts were tested within 1-2 weeks of preparation although most of the active extracts were found to retain activity after storage at 4° C. for several additional weeks. An aliquot of each extract was dried under vacuum and the dry weight of the water soluble substances extracted from each herb determined.

### Preparative Example 2

### Preparation of BZL101 for Human In Vivo Experiments

BZL101 is an aqueous extract of the aerial part of Scutellaria Barbata D. Don of the Lamiaceae family. Herba Scutellaria Barbata D. Don (Chinese pin yin transliteration—Ban Zhi Lian (BZL)) is grown mainly in areas southeastern of the Yellow River (Huang Po) in the provinces of Sichuan, Jiangsu, Jiangxi, Fujian, Guangdong, Guangxi and Shaanxi. The plant is harvested in late summer and early autumn after it blooms. The aerial part (leaves and stems) is cut from the root and is used as starting material (BZL). The aerial part of the herb is dried in the sun, packed as a whole plant. The herb is identified and verified through botanical, morphological and chemical characteristics to ensure purity.

A single dose of BZL101 is made through the following procedure and is termed BZL101 (Bionovo, Inc., Emeryville, Calif.).
180 grams of the raw herb is ground to fine powder (25 mesh)
The powder is mixed with 1800 ml of distilled water to form a slurry
The slurry is than simmered at 70-72° C. for 60 minutes
The extract is decanted and filtered through 22 μm filter
The supernatant weight after extraction is 168 gm
The volume of the solution is 1750 ml
The extract is concentrated with a vacuum evaporator to reduce the volume of water to 350 ml which constitutes a 5:1 concentration of the original solution
The dry weight of soluble material in the extract is 12 gm
It is packaged in a sterile, vacuum sealed container
Testing for bacteria, yeast and heavy metals are preformed by an accredited laboratory

### Comparative Example 1

### In vitro Inhibition of Cancer Cell Activity Cell Lines and Culture

The extract obtained in Preparative Example 1, above, was tested against four human breast cancer cell lines, SKBR3, MFC-7, MA-MB231 and BT474, and one murine breast cancer cell line, MCNeuA. All lines were maintained in 90% DME supplement with 2.0 mom L-glutamine, 100 IU/ml penicillin, 100 μg/ml streptomycin and 10% heat-inactivated fetal bovine serum. Cells at 70-80% confluence were used for plating for growth inhibition assays.

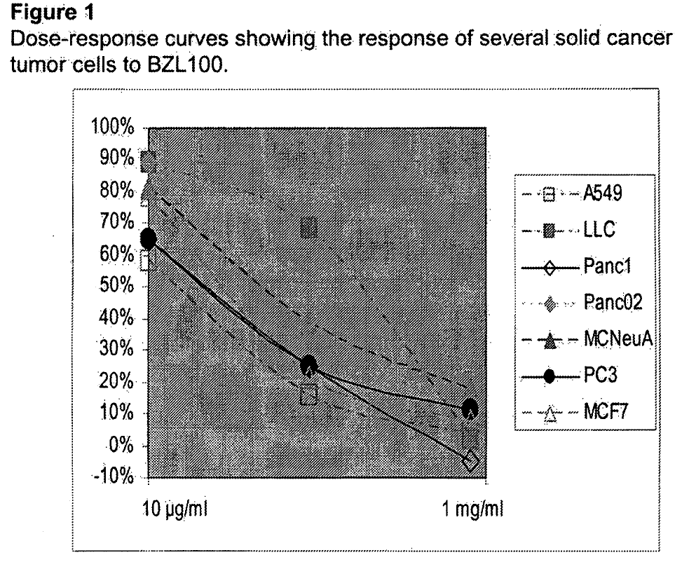

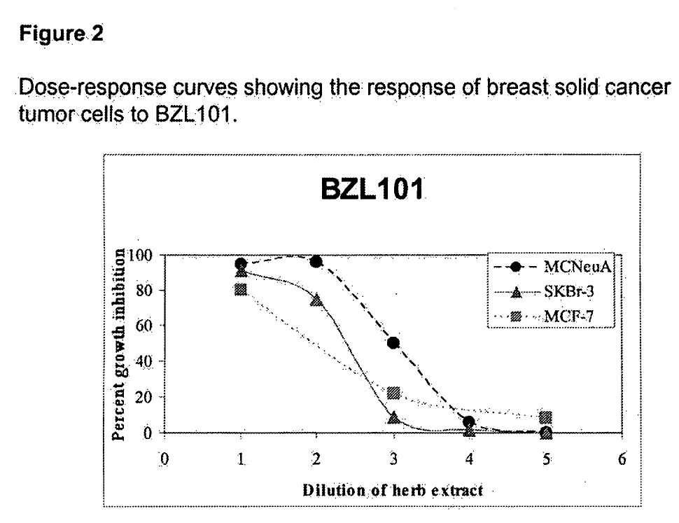

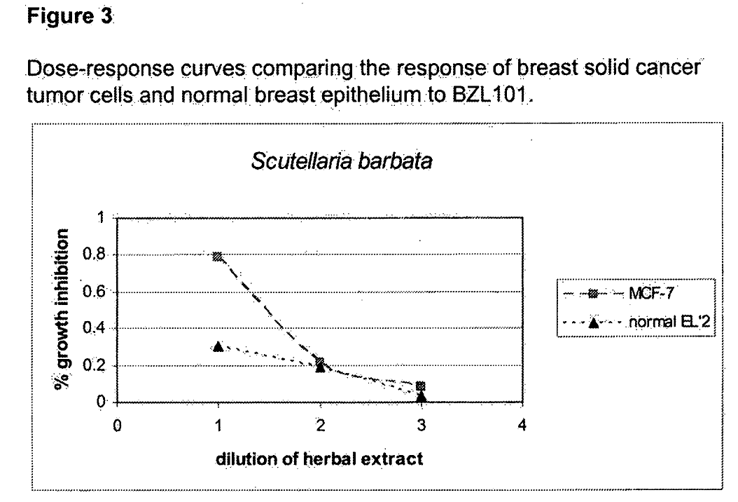

Cells were plated in 96-well flat bottom plates at 5,000 to 10,000 cells/well. The difference in number of cells plated adjusts for differences in the growth rates of these cell lines. Cells were allowed to adhere to the well walls overnight; then the extracts were added to triplicate wells at a 1:10 final dilution in culture medium for initial screening. For generating dose-response curves, serial 3-fold dilutions, starting at 1:10 dilution over 6 rows of wells were used. Water was added to the control wells at 1:10 dilution in culture medium. The plates were incubated at 37° C., 5% CO₂, for 3 days and then assayed for growth inhibition using a crystal violet assay (Bernhardt, G., et al., Standardized Kinetic Microassay to Quantify Differential Chemosensitivity on the Basis of Proliferative Activity, 1992, J. Cancer Res. Clin. Oncol., 118:35-43). Cells remaining adherent to the well walls were rinsed with PBS, the fixed cells were stained with 0.02% aqueous crystal violet (50 μl/well) for 30 minutes after which the wells were washed thoroughly with distilled water. The crystal violet stain bound by the cells was solubilized in 79% ethanol (100 μl/well) and the plates analyzed on a microplate reader (Molecular Devices) ay 595 nm. The percent inhibition was calculated as the average optical density of the control wells minus average optical density extract well divided by the average optical density of the control wells. Dose-response curves on SKBR3, MCF7 and MCNeuA cells for several of the extracts are shown in FIGS. 1-3. As can be seen, the concentration at which the extracts inhibited the activity of the cells by 50% (the IC50) ranged from over 1 mg/ml down to about 10 μg/ml.

Induction of Apoptosis

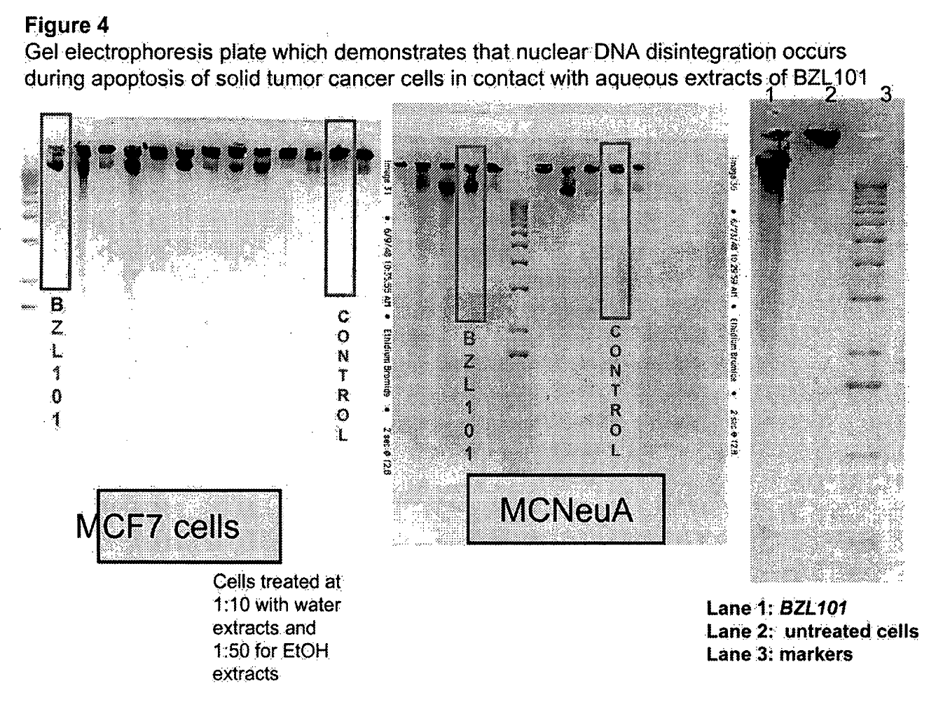

To assay for DNA fragmentation as a marker of apoptosis, a procedure for the isolation of genomic DNA that allows for the analysis of both high and low molecular weight DNA fragmentation during apoptosis was used. MCNeuA cells were plated at 5×10⁵ cells/well in 6-plates and allowed to adhere overnight. Aqueous herbal extracts were added to each well at a 1:10 and a 1:50 dilution. Sterile water, diluted 1:10 in culture medium, was added to the control wells. After 24 hours, the cells were visually examined under a microscope and morphological changes noted. Attached and floating cells were harvested, washed with cold PBS and embedded in lysis buffer (50 mM NaCl, 20 mM Tris HCl, pH 8.0, 20 mM EDTA, 0.5% sodium sarkosyl, 50 μg/ml Rnase A and 100 μg/ml proteinase K) for 1 hour at 37° C. The cells were then washed with PBS and distilled water and placed in the wells of a conventional 1% agarose gel and electrophoresed overnight at approximately 1 V/cm. The gels were then stained with ethidium bromide and photographed under UV transillumination to give intense images. The images obtained are shown in FIG. 4.

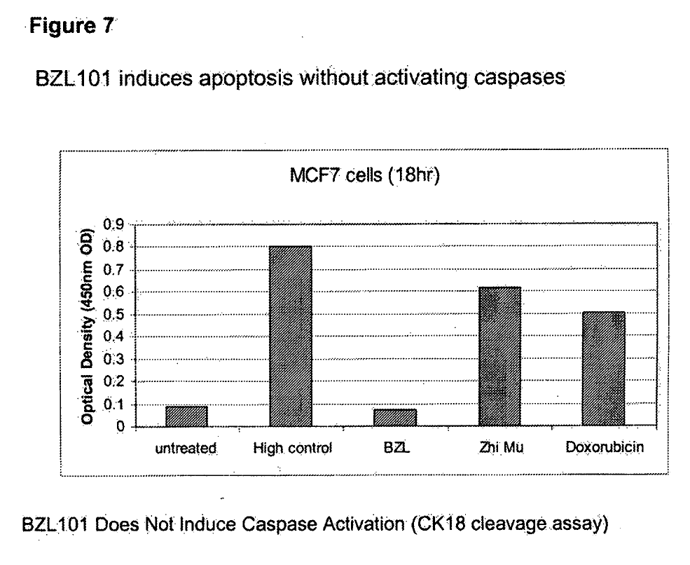

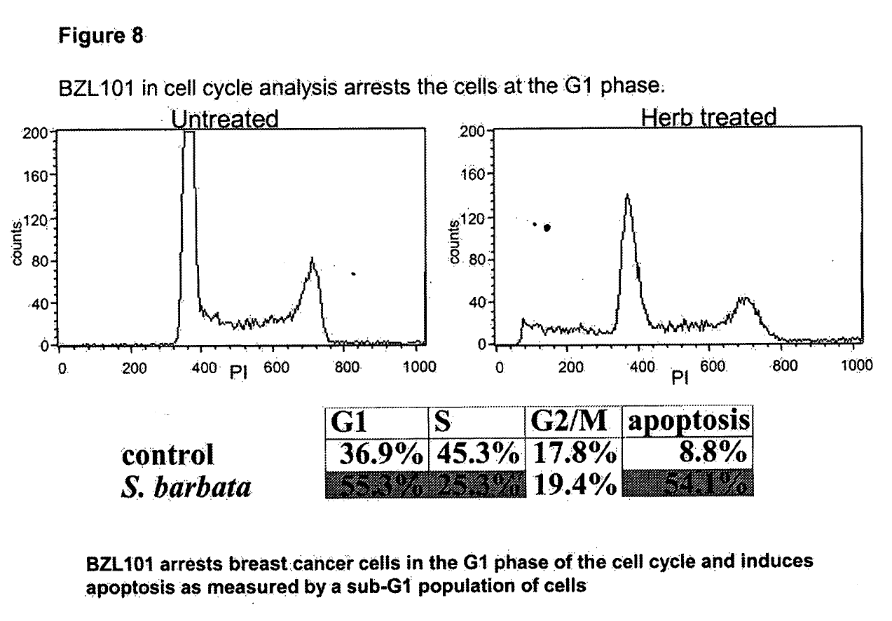

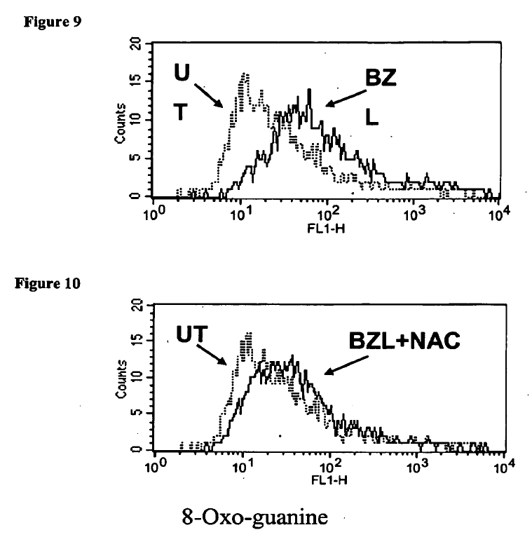

BZL101 was evaluated for antiproliferative activity on five breast cancer cell lines (SK-BR-3, MCF7, MDA-MB-231, BT474, and MCNeuA). These cell lines represent important prognostic phenotypes of breast cancer expressing a range of estrogen and HER2 receptors. BZL101, tested at a 1:10 dilution (15 μg/ml), demonstrated >50% growth inhibition on four of the five cell lines (Campbell, 2002). BZL101 showed >50% growth inhibition on a panel of lung, prostate and pancreatic cancer cell lines. BZL101 at the same dose did not cause >25% of growth inhibition on normal human mammary cells (HuMEC), demonstrating selectivity to cancer cells (Table 3). Moreso, BZL101 had a mild mitogenic effect on normal human lymphocytes. In cell cycle analysis, BZL101 caused an S phase burst and G1 arrest. (See FIG. 8). BZL101 also attenuated mitochondrial membrane potential causing caspase-independent high molecular grade (HMG) apoptosis. (See FIG. 7).

The results of this in vitro experiment are summarized in Table 3, below.

TABLE 3

| Lung | | Pancreas | | Prostate | | Breast | | | | | |
|---|---|---|---|---|---|---|---|---|---|---|---|
| A549 | LLC | Panc-1 | Panc-2 | PC-3 | LNCaP | MCF7 | BT474 | SKBR3 | MDA-MB-231 | MCNeuA | HuMEC |
| + | + | + | ++ | + | + | ++ | + | ++ | + | ++ | − |

Table 3: In vitro growth inhibitory effect of BZL101 aqueous extract of Scutellaria Barbata 1:10 dilution − <50% inhibition, + 51-75% inhibition, ++ >75% inhibition. BZL is active on all cancer cell lines but is not active on HuMECs.

### Example 1

### In vivo (IP) Efficacy of BZL101 in a Mouse Xenograft Model

In order to demonstrate the efficacy of BZL101 in the in vivo treatment of cancer, BZL101 was evaluated in a mouse xenograft model.

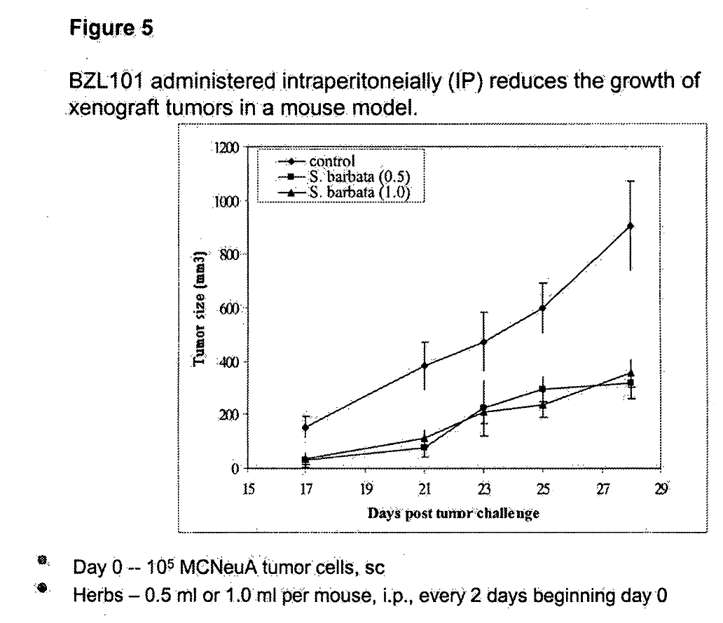

BZL101 was active via intraperitoneal (IP) administration in preventing tumor formation in a mouse xenograft model (FIG. 5). BZL101 was prepared as described in Preparative Example 1, above. Cells (10⁵) of MCNeuA cells were injected subcutaneously into mice on day 0. BZL101 (0.5 ml or 1.0 ml) or control was administered to each mouse IP every two days. Tumor size (mm³) was estimated on the 17^{th}, 21^{st}, 23^{rd}, 25^{th}, and 28^{th} day post administration. The results of this study, show in FIG. 5, demonstrate that BZL101 inhibited xenograft, suggesting that BZL101 can be an effective treatment for solid tumors in vivo.

### Example 2

### In vivo (Oral) Efficacy of BZL101 in a Mouse Xenograft Model

In order to further evaluate the effect of the herb extract in vivo, BZL101 alone, BZL101 plus cyclophosphamide and cyclophosphamide alone were orally administered to mice having subcutaneous cancer xenografts.

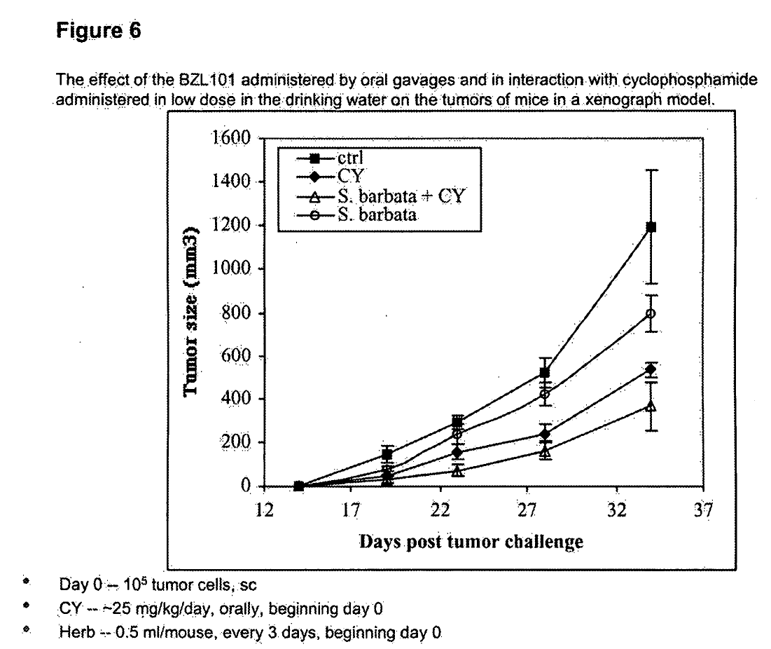

As in Example 1, above, 10⁵ cells were administered to each animal subcutaneously on Day 0. The animals were divided into four groups. The control group received only normal drinking water. The cyclophosphamide only group received 25 mg/Kg/day of cyclophosphamide in their drinking water. The BZL101 only group received 0.5 ml of BZL101 by oral gavage on Day 0 and every third day after that. The combination group received 0.5 ml/day BZL101 by oral gavage on Day zero and every third day after that, as well as 25 mg/Kg/day of cyclophosphamide in their drinking water. The results of this experiment are shown in FIG. 6.

From the results in FIG. 6, it can be seen that, as expected, cyclophosphamide alone inhibited tumor growth as compared to the control. BZL101 alone also demonstrated tumor growth inhibition. And the combination of BZL101 and cyclophosphamide inhibited tumor growth to a greater extent than did either BZL101 or cyclophosphamide alone. These results demonstrate in vivo efficacy of BZL101 in the treatment of solid tumors and suggest that BZL101 is probably effective in the treatment of solid tumors in general.

### Example 3

### Efficacy of BZL101 in Humans

In order to demonstrate the safety and clinical activity of oral BZL101, an aqueous extract from Scutellaria Barbata D. Don was studied in human patients with advanced breast cancer.

Eligible patients had histologically confirmed metastatic breast cancer and measurable disease. Patients did not receive any other chemotherapy, hormone therapy or herbal medicine during the trial. Patients received 350 ml (equivalent to 12 grams dry solubles BZL) BZL101 extract per day until disease progression, toxicity or personal preference caused them to discontinue. The primary endpoints were safety, toxicity and tumor response.

Twenty-one patients were enrolled and received BZL101. Mean age was 54 years (30-77) and mean number of prior treatments was 3.9 (0-10). There were no hematologic, nor grade III or IV non-hematologic, adverse events (AEs). Some patients reported grade I and II adverse events, such as nausea, diarrhea, headache, flatulence, vomiting, constipation, and fatigue. Sixteen patients were evaluable for response. Four of the 16 patients had stable disease (SD) for >90 days (25%) and 3/16 had SD for >180 days (19%). Five patients had minor objective tumor regression, one of which was 1 mm short of a PR based on RECIST criteria.

Patients were enrolled at the University of California, San Francisco Carol Franc Buck Breast Care Center and the Cancer Research Network in Plantation, Fla. between August 2001 and November 2004 and signed an informed consent approved by local institutional review boards. All patients were ≧18 years old with histologically confirmed diagnosis of breast cancer and clinical evidence of metastatic involvement. Patients with solitary metastases required biopsy confirmation of metastatic disease. All patients had completed prior therapies and had adequate time to recover sufficiently from the toxicities associated with prior anticancer treatments. A life expectancy of 6 months and Karnofsky performance status of 80% or better was required. Nutritional or up to five times recommended daily allowance (RDA) vitamin supplementation were permitted; but concomitant use of non-study herbal agents was prohibited. Patients were excluded from the study for the following: extensive liver involvement (>50% of liver parenchyma), lymphangitic pulmonary involvement, central nervous system involvement or spinal cord compression not stabilized by therapy for >3 months, a history of multiple or severe food or medicine allergies and organ or marrow dysfunction as defined by creatinine >2.0 mg/dl, total bilirubin >1.7 mg/dl, white blood cell count <2,500 cells/mL and platelet count <75,000 mm³.

Safety monitoring was done on a continuous basis and patients were seen by a physician for examination at baseline at every Y weeks. Adverse events were graded using Common Toxicity Criteria version 2, assigned a category by organ system and coded in relation to study drug as remote, possible, probably or definitely related. Baseline tumor assessments were done within 14 days of initiation of study drug and every three months. Responses were assessed using RECIST criteria. Study drug was administered at every visit, and at this visit compliance and a review of dosages taken was performed. BZL101 extract was provided as a liquid in a sealed and labeled aluminum packet containing a full daily dose that was administered in a split dose twice a day. Daily BZL extract was administered until the determination of tumor progression or dose limiting toxicity was encountered, or until the subject decided to voluntarily discontinue, in which case, the reason for discontinuation was obtained.

### Results

Patient Characteristics

A total of 22 patients with advanced breast cancer consented to the study and 21 patients were treated with at least one dose of oral BZL101 and included in the safety analysis. The last patient accrued to the study was not treated with BZL101 as funding for the study from the California Breast Cancer Research Program had ended and the expiration date for the study medication was nearing. Sixteen of the patients were treated for 28 days or more and evaluable according to the Response Evaluation Criteria in Solid Tumors (RECIST). Nine subjects discontinued study medication due to patient preference, and twelve patients were removed from the study due to progression based on RECIST criteria. None of the patients were removed from the study due to either grade III or IV adverse events categorized according to the National Cancer Institute (NCI) Common Toxicity Criteria (CTC) version 2. See Table 4 for a summary of study participants and Table 5 for a summary of selected patient characteristics.

TABLE 4
Summary of Study Participants

| | |
|---|---|
| Study Participants Consented | 22 |
| Consented but not Treated with BZL101 | 1* |
| Included in Safety Analysis | 21 |
| Evaluable by RECIST Criteria | 16 |
| Off Study Due to Patient Preference | 9 |
| Off Study Due to Progression of Disease | 12 |
| Off Study Due to Grade III or IV Toxicity | 0 |

*Inventory of study medication was nearing expiration and funding for the study had ended.

TABLE 5
Summary of Baseline Characteristics: Age,
Height, Weight, Race or Ethnicity

| Age | | |
|---|---|---|
| Mean | 54.3 | years |
| Median | 55.5 | years |
| Range | 30-77 | years |
| Height | | |
| Mean | 65.2 | inches |
| Median | 65.0 | inches |
| Range | 62-68 | inches |
| Weight | | |
| Mean | 137.1 | pounds |
| Median | 139 | pounds |
| Range | 108-165 | pounds |
| Race or Ethnicity | | |
| Caucasian | 13 | (59%) |

| | |
|---|---|
| African American | 2 participants (9%) |
| Hispanic | 1 participant (5%) |
| Asian | 1 participant (5%) |
| Native American | 1 participant (5%) |
| Unknown | 4 participants (18%) |

Safety Data

There were no deaths, serious adverse events or hematological adverse effects attributed to the study medication BZL101. There were no grade III or IV toxicities that were classified as possibly, probably or definitely related to BZL101.

Efficacy

Of the 21 patients who were treated with study medication, 16 patients were on the trial for 28 days or more and evaluable for response. Four of the 16 patients (25%) had stable disease for >90 days and 3/16 (19%) had stable disease for >180 days. Five patients had some degree of objective tumor regression, classified as a minimal response (<10% but <30 reduction in diameter sums). One of these responses was 1 mm short of a partial remission based on RECIST criteria. The average number of prior therapies for metastatic disease prior to treatment with the study medication, for patients who took at least one dose of BZL101, was 3.9 (See Table 6).

TABLE 6
Response to Treatment Based on RECIST Criteria

| | | | | | Prior Therapies After | | | | | | |
|---|---|---|---|---|---|---|---|---|---|---|---|
| Patient | | | Days on | Reason for | Diagnosis of Metastasis | | | | | | |
| # | Age | On Study | Study | Discontinuation | But Before BZL101 | NE | PD | SD | PR | CR | MR |
| 2001 | 48 | Aug. 28, 2001- | 184 | Progression | CMF Capecitabine | | 6 | 3 | | | |
| | | Mar. 14, 2002 | | | | | | | | | |
| 2002 | 30 | Oct. 02, 2001- | 25 | Progression | Goserelin Anastrozole Tamoxifen | | <1 | | | | |
| | | Oct. 26, 2001 | | | Targretin trial Docetaxel AC | | | | | | |
| | | | | | High dose chemo Capecitabine | | | | | | |
| | | | | | VEGF Trial Exemestane | | | | | | |
| 2003 | 50 | Oct. 30, 2001- | 151 | Pt | Anastrozole Tamoxifen | | | 5 | | | 2, 3, 4 |
| | | Apr. 17, 2002 | | Preference | | | | | | | |
| 2004 | 77 | Dec. 20, 2001- | 259 | Progression | None | | 9 | 6 | | | 3 |
| | | Sep. 05, 2002 | | | | | | | | | |
| 2005 | 64 | Mar. 07, 2002- | 36 | Pt | None | | | 1 | | | |
| | | Apr. 11, 2002 | | Preference | | | | | | | |
| 2006 | 59 | Oct. 31, 2002- | 71 | Pt | CAF Tamoxifen CMF Paclitaxel | NE | | | | | |
| | | Jan. 09, 2003 | | preference | Carboplatin + Etoposide | | | | | | |
| | | | | | Capecitabine | | | | | | |
| 2007 | 60 | Dec. 09, 2002- | 16 | Pt | Docetaxel Trastuzamab Cisplatin | NE | | | | | |
| | | Dec. 25, 2002 | | Preference | Capicitabine Liposomal doxirubicin | | | | | | |
| | | | | | Gemcitabine | | | | | | |
| 2008 | 52 | Jun. 24, 2003- | 59 | Pt | Exemestane Tamoxifen Capecitabine | NE | | | | | |
| | | Aug. 21, 2003 | | Preference | | | | | | | |
| 2009 | 34 | Sep. 12, 2003- | 41 | Progression | Doxorubicin Paciltaxel Docetaxel | | 1.5 | | | | |
| | | Oct. 28, 2003 | | | | | | | | | |
| 2010 | 56 | Jun. 26, 2003- | 1 | Pt | Tamoxifen CAF Traztuzamab | NE | | | | | |
| | | Jun. 27, 2003 | | Preference | Gemcitabine Letrozole Fulvestrant | | | | | | |
| 2011 | 48 | Apr. 21, 2004- | 93 | Progression | Docetaxil Gemcitabine | | 3 | | | | |
| | | Jul. 23, 2004 | | | | | | | | | |
| 2012 | | Nov. 08, 2004- | 6 | Pt | Letrozole Fulvestrant | NE | | | | | |
| | | Nov. 15, 2004 | | Preference | Carboplatin + Docetaxel | | | | | | |
| | | | | | Zoledronic acid | | | | | | |
| 3001 | 54 | Feb. 28, 2002- | 51 | Progression | Vinorelbine Traztuzamab | | 1.5 | | | | |
| | | Apr. 19, 2002 | | | Capecitabine | | | | | | |
| 3002 | 48 | Feb. 28, 2002- | 7 | Pt | Anastrazole Letrazole | NE | | | | | |
| | | Mar. 07, 2002 | | Preference | | | | | | | |
| 3003 | 59 | Mar. 01, 2002- | 260 | Progression | Liposomal doxorubicin + | | 9 | | | | 1 |
| | | Nov. 15, 2002 | | | Paclitaxel | | | | | | |
| 3004 | 59 | Mar. 04, 2002- | 33 | Progression | Tamoxifen Docetaxel Letrazole | | | | | | 1 |
| | | Apr. 06, 2002 | | | | | | | | | |
| 3005 | 60 | Mar. 29, 2002- | 42 | Progression | Tamoxifen Letrozole Anastrozole | | 1 | | | | |
| | | May 12, 2002 | | | Vinorelbine + Capecitabine NFL | | | | | | |
| 3006 | 56 | Apr. 17, 2002- | 63 | Progression | Tamoxifen Liposomal doxorubicin | | 2 | | | | 1 |
| | | Jul. 01, 2002 | | | NFL Anastrozole Trastuzamab | | | | | | |
| | | | | | Vinorelbine Gemcitabine Capecitabine | | | | | | |
| 3007 | 54 | Sep. 13, 2002- | 59 | Progression | TAC Tamoxifen Doxorubicin Trastuzamab | | 2 | | | | |
| | | Nov. 11, 2002 | | | Docetaxel CMF Vinorelbine Capecitabine | | | | | | |
| | | | | | Fulvestrant | | | | | | |
| 3008 | 67 | Apr. 09, 2004- | 38 | Pt | Paclitaxel Vinorelbine + Capecitabine | | | 1 | | | |
| | | May 17, 2004 | | Preference | Pfizer clinical trial Docetaxel | | | | | | |
| | | | | | Gemcitabine Liposomal doxorubicin | | | | | | |
| 3009 | 45 | May 24, 2004- | 95 | Progression | None | | 3 | | | | |
| | | Aug. 27, 2004 | | | | | | | | | |
| 3010 | 59 | Not treated | 0 | | Tamoxifen Anastrozole Capecitabine | NE | | | | | |
| | | | | | Vinorelbine Liposomal doxorubicin + | | | | | | |
| | | | | | Gemcitabine Carboplatin + Paclitaxel | | | | | | |
| | | | | | Fulvestrant Toremifene Letrozole | | | | | | |
| | | | | | Zoledronic Acid | | | | | | |

Recist Criteria (Months)
NE = Not evaluable
PD = Progressive Disease,
SD = Stable Disease,
PR = Partial Remission,
CR = Complete Remission
MR = Minimal Response, >0% and <30%reduction
NFL mitoxantrone, 5-fluorouracil, leucovorin
CMF cyclophosphamide, methotrexate, fluorouracil
CAF cyclophosphamide, adriamycin, 5-fluorouracil
TAC docetaxel, adriamycin (doxorubicin), cyclophosphamide
AC adriamycin (doxorubicin), cyclophosphamide

In a modified RECIST evaluation, where all measurable lesions were included as evaluable, one patient had a partial response or a reduction of 31% in the sum of the longest tumor diameter of all measurable lesions after 7 weeks of treatment and a reduction of 33% after 11 weeks of treatment (Table 7).

TABLE 7
Patient #2003 Response to Treatment Based on Modified RECIST Criteria

| | Lesion 1 | Lesion 2 | Lesion 3 | Lesion 4 | |
|---|---|---|---|---|---|
| | Site and | Site and | Site and | Site and | Total |
| | Method | Method | Method | Method | Measurable |
| DATE | Measurement | Measurement | Measurement | Measurement | Disease |
| #2003 | Site: Lymph | Site: Lymph | Site: Lymph | Site: | Total Baseline |
| Baseline | Node-Left | Node-Anterior | Node-Left | Vertebrae/Pelvis | Diameters = |
| Oct. 30, 2001 | Subclavian | Cervical | Subclavian, Post | Method: Pelvic | 5.8 cm |
| | Method: | Method: | Cervical | CT scan | |
| | Palpation | Palpation | Method: | Bony metastases | |
| | Measurement: | Measurement: | Palpation | | |
| | 3.0 × 2.5 cm | 2.0 × 2.0 cm | Measurement: | | |
| | | | 0.8 cm | | |
| Month 2 | Measurement: | Measurement: | Measurement: | Site: Bone | Total Sum = |
| Dec. 20, 2001 | 2.0 × 2.0 cm | 1.5 × 1.0 cm | 0.5 cm | Method: Bone | 4.0 cm |
| | | | | Scan | % Change = −31% |
| | | | | Bony Mets | |
| Month 3 | Measurement: | Measurement: | Measurement: | Site: Bone | Total Sum = |
| Jan. 22, 2002 | 2.1 × 1.5 cm | 1.5 × 1.2 cm | 0.3 cm | Method: Bone | 3.9 cm |
| | | | | Scan | % Change = −33% |
| | | | | Bony mets | |
| | | | | grossly stable | |
| | | | | compared with | |
| | | | | Nov. 19, 2001 | |
| Month 4 | Measurement: | Measurement: | Measurement: | | Total Sum = |
| Mar. 08, 2002 | 2.0 × 1.5 cm | 2.0 × 2.0 cm | 0.5 cm | | 4.5 cm |
| | | | | | % Change = −24% |
| Month 5 | Measurement: | Measurement: | Measurement: | | Total Sum = |
| Apr. 17, 2002 | 3.0 × 2.5 cm | 2.0 × 1.5 cm | 0.5 cm | | 5.5 cm |
| | | | | | % Change = −5% |

### CONCLUSION

The herbal extract BZL101, its uses for the inhibition of solid tumor cancer cells and the treatment of such cancers in patients are described herein. Although certain embodiments and examples have been used to describe the present invention, it will be apparent to those skilled in the art that changes to the embodiments and examples may be made without departing from the scope and spirit of this invention.

### Example 4

### Efficacy of BZL101 in Humans

In order to demonstrate the safety and clinical activity of oral BZL101, an aqueous extract from Scutellaria Barbata D. Don is studied in human patients with advanced breast cancer.

Eligible patients have histologically confirmed metastatic breast cancer and measurable disease. Patients do not receive any other chemotherapy, hormone therapy or herbal medicine during the trial. Patients receive 350 ml (dry residue from 180 g BZL; approximately 12 grams dry soluble BZL extract) concentrated BZL101 extract per day until disease progression, toxicity or personal preference caused them to discontinue. The primary endpoints are safety, toxicity and tumor response.

Patients meeting one or more of the following criteria are enrolled:

Advanced (metastatic) breast cancer
Nuclear estrogen receptor (ER) negative—i.e. the cancer expresses ER at a level that does not exceed a predetermined threshold (lower limit)
Nuclear estrogen receptor (ER) positive—i.e. the cancer expresses ER at a level that exceeds a predetermined threshold (lower limit)

Early stage (non-metastatic) breast cancer
Nuclear estrogen receptor (ER) negative—i.e. the cancer expresses ER at a level that does not exceed a predetermined threshold (lower limit)
Nuclear estrogen receptor (ER) positive—i.e. the cancer expresses ER at a level that exceeds a predetermined threshold (lower limit)

ER status (ER⁺ or ER⁻) is determined by accepted methods, e.g. by fluoroscopically or isotopically labeled antibody assay or gene chip analysis. Cancer grade is determined by methods known to the clinical oncologist, such as by histological methods known in the art.

Patients are classified as early stage (i.e. non-metastatic) or advanced (metastatic) and are enrolled and are treated with BZL alone or with BZL in combination with another chemotherapeutic agent according to the following schedule:

| ER | Cancer | | | Second Combo |
|---|---|---|---|---|
| Status | Grade | BZL101 | First Combo Drug | Drug |
| + | Advanced | 1× | — | — |
| + | Advanced | 2× | — | — |
| + | Advanced | 4× | — | — |
| + | Advanced | 8× | — | — |
| − | Advanced | 1× | — | — |
| − | Advanced | 2× | — | — |
| − | Advanced | 4× | — | — |
| − | Advanced | 8× | — | — |
| + | Advanced | 1× | armidex | — |
| − | Advanced | 1× | armidex | — |
| + | Advanced | 1× | aramosin | — |
| − | Advanced | 1× | aramosin | — |
| + | Advanced | 1× | letrozole | — |
| − | Advanced | 1× | letrozole | — |
| + | Advanced | 1× | doxorubicin | — |
| − | Advanced | 1× | doxorubicin | — |
| + | Advanced | 1× | cyclophosphamide | — |
| − | Advanced | 1× | cyclophosphamide | — |
| + | Advanced | 1× | doxorubicin | cyclophosphamide |
| − | Advanced | 1× | doxorubicin | cyclophosphamide |
| + | Advanced | 1× | paclitaxel | — |
| − | Advanced | 1× | paclitaxel | — |
| + | Advanced | 1× | docetaxel | — |
| − | Advanced | 1× | docetaxel | — |
| + | Advanced | 1× | capecitabine | — |
| − | Advanced | 1× | capecitabine | — |
| + | Advanced | 1× | gemcitabine | — |
| − | Advanced | 1× | gemcitabine | — |
| + | Advanced | 1× | methotrexate | — |
| − | Advanced | 1× | methotrexate | — |
| + | Advanced | 1× | 5FU | — |
| − | Advanced | 1× | 5FU | — |
| + | Advanced | 1× | cyclophosphamide | 5FU/methotrexate |
| − | Advanced | 1× | cyclophosmamide | 5FU/methotrexate |
| + | Advanced | 1× | trastuzumab | — |
| − | Advanced | 1× | trastuzumab | — |
| + | Advanced | 1× | bevacizumab | — |
| − | Advanced | 1× | bevacizumab | — |
| + | Advanced | 1× | lapatinib | — |
| − | Advanced | 1× | lapatinib | — |
| + | Early | 1× | — | — |
| + | Early | 2× | — | — |
| + | Early | 4× | — | — |
| + | Early | 8× | — | — |
| − | Early | 1× | — | — |
| − | Early | 2× | — | — |
| − | Early | 4× | — | — |
| − | Early | 8× | — | — |
| + | Early | 1× | armidex | — |
| − | Early | 1× | armidex | — |
| + | Early | 1× | aramosin | — |
| − | Early | 1× | aramosin | — |
| + | Early | 1× | letrozole | — |
| − | Early | 1× | letrozole | — |
| + | Early | 1× | doxorubicin | — |
| − | Early | 1× | doxorubicin | — |
| + | Early | 1× | cyclophosphamide | — |
| − | Early | 1× | cyclophosphamide | — |
| + | Early | 1× | doxorubicin | cyclophosphamide |
| − | Early | 1× | doxorubicin | cyclophosphamide |
| + | Early | 1× | paclitaxel | — |
| − | Early | 1× | paclitaxel | — |
| + | Early | 1× | docetaxel | — |
| − | Early | 1× | docetaxel | — |
| + | Early | 1× | capecitabine | — |
| − | Early | 1× | capecitabine | — |
| + | Early | 1× | gemcitabine | — |
| − | Early | 1× | gemcitabine | — |
| + | Early | 1× | methotrexate | — |
| − | Early | 1× | methotrexate | — |
| + | Early | 1× | 5FU | — |
| − | Early | 1× | 5FU | — |
| + | Early | 1× | cyclophosphamide | 5FU/methotrexate |
| − | Early | 1× | cyclophosmamide | 5FU/methotrexate |
| + | Early | 1× | trastuzumab | — |
| − | Early | 1× | trastuzumab | — |
| + | Early | 1× | bevacizumab | — |
| − | Early | 1× | bevacizumab | — |
| + | Early | 1× | lapatinib | — |
| − | Early | 1× | lapatinib | — |
| + | Early | 1× | tamoxifen | — |
| − | Early | 1× | tamoxifen | — |
| + | Early | 1× | raloxifene | — |
| − | Early | 1× | raloxifene | — |

“Advanced” tumors are metastatic
“Early” tumors are non-metastatic
Multipliers (1×, 2×, etc.) indicate the amount of BZL101 given. BZL101 is a composition comprising the dry solid residue of an extract of 180 g of Scutellaria barbata D. Don (BZL); 1× indicates that the dry solid residue of an extract of 180 g of BZL is administered per day; thus 2× would be the dry solid residue of 360 g of BZL, and so forth.

Safety monitoring is done on a continuous basis and patients are seen by a physician for examination at baseline at regular intervals. Adverse events are graded using Common Toxicity Criteria version 2, assigned a category by organ system and coded in relation to study drug as remote, possible, probably or definitely related. Baseline tumor assessments are done within 14 days of initiation of study drug and every three months. Responses are assessed using RECIST criteria. Study drugs are administered at every visit, and at this visit compliance and a review of dosages taken is performed. BZL101 extract is provided as a liquid in a sealed and labeled aluminum packet containing a full daily dose that is administered in a split dose twice a day. Daily BZL extract is administered until the determination of tumor progression or dose limiting toxicity is encountered, or until the subject decided to voluntarily discontinue, in which case, the reason for discontinuation is obtained. Additional chemotherapeutic agents, when administered, are administered according to established procedures for the specific drugs. In some instances, some fraction of the minimum effective dose is administered (e.g. about 0.1× to about 0.8× the normal minimum effective dose).

### Example 5

### Efficacy of BZL101 in Treatment-Refractory Breast Cancer in Humans

In order to demonstrate the efficacy of oral BZL101 in patients previously treated with another chemotherapeutic agent to which the cancer proved to be refractory, an aqueous extract from Scutellaria Barbata D. Don is studied in human patients.

Eligible patients have histologically confirmed metastatic breast cancer and measurable disease. Patients do not receive any other chemotherapy, hormone therapy or herbal medicine during the trial. Patients receive 350 ml (dry residue from 180 g BZL; approximately 12 grams dry soluble BZL extract) concentrated BZL101 extract per day until disease progression, toxicity or personal preference caused them to discontinue. The primary endpoints are safety, toxicity and tumor response. Each patient will have been previously treated with at least one other chemotherapeutic agent that will have proven to be refractory toward treatment with that chemotherapeutic agent.

Patients meeting one or more of the following criteria are enrolled:

Advanced (metastatic) breast cancer
Nuclear estrogen receptor (ER) negative—i.e. the cancer expresses ER at a level that does not exceed a predetermined threshold (lower limit)
Nuclear estrogen receptor (ER) positive—i.e. the cancer expresses ER at a level that exceeds a predetermined threshold (lower limit)

Early stage (non-metastatic) breast cancer
Nuclear estrogen receptor (ER) negative—i.e. the cancer expresses ER at a level that does not exceed a predetermined threshold (lower limit)
Nuclear estrogen receptor (ER) positive—i.e. the cancer expresses ER at a level that exceeds a predetermined threshold (lower limit)

ER status (ER⁺ or ER⁻) is determined by accepted methods, e.g. by fluoroscopically or isotopically labeled antibody assay or gene chip analysis. Cancer grade is determined by methods known to the clinical oncologist, such as by histological methods known in the art.

Patients are classified as early stage (i.e. non-metastatic) or advanced (metastatic) and are enrolled and are treated with BZL alone or with BZL in combination with another chemotherapeutic agent according to the following schedule:

| | | | | Previously |
|---|---|---|---|---|
| | | | Previously | Administered |
| | | | Administered Drug | Drug (Second and |
| ER Status | Cancer Grade | BZL101 | (First) | Subsequent) |
| + | Advanced | 1× | armidex | — |
| + | Advanced | 2× | armidex | — |
| + | Advanced | 4× | armidex | — |
| + | Advanced | 8× | armidex | — |
| − | Advanced | 1× | armidex | — |
| − | Advanced | 2× | armidex | — |
| − | Advanced | 4× | armidex | — |
| − | Advanced | 8× | armidex | — |
| + | Advanced | 1× | aramosin | — |
| + | Advanced | 2× | aramosin | — |
| + | Advanced | 4× | aramosin | — |
| + | Advanced | 8× | aramosin | — |
| − | Advanced | 1× | aramosin | — |
| − | Advanced | 2× | aramosin | — |
| − | Advanced | 4× | aramosin | — |
| − | Advanced | 8× | aramosin | — |
| + | Advanced | 1× | letrozole | — |
| + | Advanced | 2× | letrozole | — |
| + | Advanced | 4× | letrozole | — |
| + | Advanced | 8× | letrozole | — |
| − | Advanced | 1× | letrozole | — |
| − | Advanced | 2× | letrozole | — |
| − | Advanced | 4× | letrozole | — |
| − | Advanced | 8× | letrozole | — |
| + | Advanced | 1×, 2×, 4× or 8× | doxorubicin | — |
| − | Advanced | 1×, 2×, 4× or 8× | doxorubicin | — |
| + | Advanced | 1×, 2×, 4× or 8× | cyclophosphamide | — |
| − | Advanced | 1×, 2×, 4× or 8× | cyclophosphamide | — |
| + | Advanced | 1×, 2×, 4× or 8× | doxorubicin | cyclophosphamide |
| − | Advanced | 1×, 2×, 4× or 8× | doxorubicin | cyclophosphamide |
| + | Advanced | 1×, 2×, 4× or 8× | paclitaxel | — |
| − | Advanced | 1×, 2×, 4× or 8× | paclitaxel | — |
| + | Advanced | 1×, 2×, 4× or 8× | docetaxel | — |
| − | Advanced | 1×, 2×, 4× or 8× | docetaxel | — |
| + | Advanced | 1×, 2×, 4× or 8× | capecitabine | — |
| − | Advanced | 1×, 2×, 4× or 8× | capecitabine | — |
| + | Advanced | 1×, 2×, 4× or 8× | gemcitabine | — |
| − | Advanced | 1×, 2×, 4× or 8× | gemcitabine | — |
| + | Advanced | 1×, 2×, 4× or 8× | methotrexate | — |
| − | Advanced | 1×, 2×, 4× or 8× | methotrexate | — |
| + | Advanced | 1×, 2×, 4× or 8× | 5FU | — |
| − | Advanced | 1×, 2×, 4× or 8× | 5FU | — |
| + | Advanced | 1×, 2×, 4× or 8× | cyclophosphamide | 5FU/methotrexate |
| − | Advanced | 1×, 2×, 4× or 8× | cyclophosmamide | 5FU/methotrexate |
| + | Advanced | 1×, 2×, 4× or 8× | trastuzumab | — |
| − | Advanced | 1×, 2×, 4× or 8× | trastuzumab | — |
| + | Advanced | 1×, 2×, 4× or 8× | bevacizumab | — |
| − | Advanced | 1×, 2×, 4× or 8× | bevacizumab | — |
| + | Advanced | 1×, 2×, 4× or 8× | lapatinib | — |
| − | Advanced | 1×, 2×, 4× or 8× | lapatinib | — |
| + | Early | 1× | armidex | — |
| + | Early | 2× | armidex | — |
| + | Early | 4× | armidex | — |
| + | Early | 8× | armidex | — |
| − | Early | 1× | armidex | — |
| − | Early | 2× | armidex | — |
| − | Early | 4× | armidex | — |
| − | Early | 8× | armidex | — |
| + | Early | 1× | aramosin | — |
| − | Early | 2× | aramosin | — |
| + | Early | 4× | aramosin | — |
| − | Early | 8× | aramosin | — |
| + | Early | 1× | aramosin | — |
| − | Early | 2× | aramosin | — |
| + | Early | 4× | aramosin | — |
| − | Early | 8× | aramosin | — |
| + | Early | 1× | letrozole | — |
| − | Early | 2× | letrozole | — |
| + | Early | 4× | letrozole | — |
| − | Early | 8× | letrozole | — |
| + | Early | 1× | letrozole | — |
| − | Early | 2× | letrozole | — |
| + | Early | 4× | letrozole | — |
| − | Early | 8× | letrozole | — |
| + | Early | 1×, 2×, 4× or 8× | doxorubicin | — |
| − | Early | 1×, 2×, 4× or 8× | doxorubicin | — |
| + | Early | 1×, 2×, 4× or 8× | cyclophosphamide | — |
| − | Early | 1×, 2×, 4× or 8× | cyclophosphamide | — |
| + | Early | 1×, 2×, 4× or 8× | doxorubicin | cyclophosphamide |
| − | Early | 1×, 2×, 4× or 8× | doxorubicin | cyclophosphamide |
| + | Early | 1×, 2×, 4× or 8× | paclitaxel | — |
| − | Early | 1×, 2×, 4× or 8× | paclitaxel | — |
| + | Early | 1×, 2×, 4× or 8× | docetaxel | — |
| − | Early | 1×, 2×, 4× or 8× | docetaxel | — |
| + | Early | 1×, 2×, 4× or 8× | capecitabine | — |
| − | Early | 1×, 2×, 4× or 8× | capecitabine | — |
| + | Early | 1×, 2×, 4× or 8× | gemcitabine | — |
| − | Early | 1×, 2×, 4× or 8× | gemcitabine | — |
| + | Early | 1×, 2×, 4× or 8× | methotrexate | — |
| − | Early | 1×, 2×, 4× or 8× | methotrexate | — |
| + | Early | 1×, 2×, 4× or 8× | 5FU | — |
| − | Early | 1×, 2×, 4× or 8× | 5FU | — |
| + | Early | 1×, 2×, 4× or 8× | cyclophosphamide | 5FU/methotrexate |
| − | Early | 1×, 2×, 4× or 8× | cyclophosmamide | 5FU/methotrexate |

“Advanced” tumors are metastatic
“Early” tumors are non-metastatic
Multipliers (1×, 2×, etc.) indicate the amount of BZL101 given. BZL101 is a composition comprising the dry solid residue of an extract of 180 g of Scutellaria barbata D. Don (BZL); 1× indicates that the dry solid residue of an extract of 180 g of BZL is administered per day; thus 2× would be the dry solid residue of 360 g of BZL, and so forth.

Safety monitoring is done on a continuous basis and patients are seen by a physician for examination at baseline at every Y weeks. Adverse events are graded using Common Toxicity Criteria version 2, assigned a category by organ system and coded in relation to study drug as remote, possible, probably or definitely related. Baseline tumor assessments are done within 14 days of initiation of study drug and every three months. Responses are assessed using RECIST criteria. Study drugs are administered at every visit, and at this visit compliance and a review of dosages taken is performed. BZL101 extract is provided as a liquid in a sealed and labeled aluminum packet containing a full daily dose that is administered in a split dose twice a day. Daily BZL extract is administered until the determination of tumor progression or dose limiting toxicity is encountered, or until the subject decided to voluntarily discontinue, in which case, the reason for discontinuation is obtained. Additional chemotherapeutic agents, when administered, are administered according to established procedures for the specific drugs. In some instances, some fraction of the minimum effective dose is administered (e.g. about 0.1× to about 0.8× the normal minimum effective dose).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a cancer, comprising:
(a) determining the level of expression of nuclear estrogen receptor (ER) in the cancer; and
(b) if the level of expression of ER is at or above a predetermined threshold administering to the patient a first treatment comprising an extract of Scutellaria Barbata D. Don;
wherein if the level of expression of FR is below a predetermined threshold administering to the patient a therapeutically effective amount of an alternate treatment.

2. The method of claim 1, wherein the cancer is breast cancer, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma

3. The method of claim 1, wherein the cancer is breast cancer.

4. The method of claim 1, further comprising administering to the patient a second treatment.

5. The method of claim 1, further comprising administering to the patient a second treatment selected from surgery, chemotherapy, and/or radiation therapy.

6. The method of claim 1, further comprising administering a second treatment selected from: an estrogen receptor modulator, an aromatase inhibitor, an antitumor antibiotic, a nitrogen mustard, a taxane, an antimetabolite, an anti-cancer monoclonal antibody, a tyrosine kinase inhibitor, or combinations thereof.

7. The method of claim 1, further comprising administering a second treatment selected from: tamoxifen, raloxifene, arimidex, aromasin, letrozole, daunorubicin, doxorubicin, epirubicin, idarubicin, mitoxantrone, valrubicin, actinomycin, bleomycin, mitomycin, plicamycin, chlorambucil, chlormethine, cyclophosphamide, ifosfamide, melphalan, paclitaxel, docetaxel, aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine, gemcitabine, alemtuzumab, bevacizumab, cetuximab, gemtuzumab, panitumumab, rituximab, tositumomab, trastuzumab, dasatinib, erlotinib, gefitinib, imatinib, lapatinib, nilotinib, sorafenib, sunitinib, or combinations thereof.

8. The method of claim 1, wherein the first therapeutic agent is administered before, after, or simultaneously with the second therapeutic agent.

9. A method of treating a cancer, comprising:
(a) determining the level of expression of nuclear estrogen receptor (ER) in the cancer; and
(b) if the level of expression of ER is at or below a predetermined threshold administering to the patient a first treatment comprising an extract of Scutellaria Barbata D. Don;
wherein if the level of expression of ER is above a predetermined threshold administering to the patient a therapeutically effective amount of an alternate treatment.

10. The method of claim 8, wherein the cancer is breast cancer, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma and retinoblastoma

11. The method of claim 8, wherein the cancer is breast cancer.

12. The method of claim 8, wherein the cancer is ERα- and/or ERβ-breast cancer.

13. The method of claim 8, wherein the breast cancer is refractory to treatment with an estrogen receptor modulator, an aromatase inhibitor, or combinations thereof.

14. The method of claim 1, further comprising administering to the patient a second treatment.

15. The method of claim 1, further comprising administering to the patient a second treatment selected from surgery, chemotherapy, and/or radiation therapy.

16. The method of claim 1, further comprising administering a second treatment selected from: tamoxifen, raloxifene, arimidex, aromasin, letrozole, daunorubicin, doxorubicin, epirubicin, idarubicin, mitoxantrone, valrubicin, actinomycin, bleomycin, mitomycin, plicamycin, chlorambucil, chlormethine, cyclophosphamide, ifosfamide, melphalan, paclitaxel, docetaxel, aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine, gemcitabine, alemtuzumab, bevacizumab, cetuximab, gemtuzumab, panitumumab, rituximab, tositumomab, trastuzumab, dasatinib, erlotinib, gefitinib, imatinib, lapatinib, nilotinib, sorafenib, sunitinib, or combinations thereof.

17. The method of claim 1, wherein the first therapeutic agent is administered before, after, or simultaneously with the second therapeutic agent.

18. A kit for treatment of cancer, comprising a therapeutically effective amount of a first chemotherapeutic agent comprising an extract of Scutellaria Barbata D. Don and a therapeutically effective amount of a second chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor.

19. The kit of claim 13, wherein the second chemotherapeutic agent is:
(a) an aromatase selected from arimidex, aromasin and letrozole;
(b) an antitumor antibiotic selected from daunorubicin, doxorubicin, epirubicin, idarubicin, mitoxantrone, valrubicin, actinomycin, bleomycin, mitomycin and plicamycin;
(c) a nitrogen mustard selected from chlorambucil, chlormethine, cyclophosphamide, ifosfamide and melphalan;
(d) a taxane selected from paclitaxel and docetaxel;
(e) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine;
(f) an anti-cancer monoclonal antibody selected from alemtuzumab, bevacizumab, cetuximab, gemtuzumab, panitumumab, rituximab, tositumomab and trastuzumab;
(g) a tyrosine kinase inhibitor selected from dasatinib, erlotinib, gefitinib, imatinib, lapatinib, nilotinib, sorafenib and sunitinib,
(h) or combinations thereof.

20. The kit of claim 13, further comprising a third chemotherapeutic agent selected from the group consisting of an aromatase inhibitor, an antitumor antibiotic, a nitrogen mustard, a taxane, an antimetabolite, an anti-cancer monoclonal antibody and a tyrosine kinase inhibitor.
(a) The kit of claim 13, wherein the third chemotherapeutic agent is:
(b) an aromatase selected from arimidex, aromasin and letrozole;
(c) an antitumor antibiotic selected from daunorubicin, doxorubicin, epirubicin, idarubicin, mitoxantrone, valrubicin, actinomycin, bleomycin, mitomycin and plicamycin;
(d) a nitrogen mustard selected from chlorambucil, chlormethine, cyclophosphamide, ifosfamide and melphalan;
(e) a taxane selected from paclitaxel and docetaxel;
(f) an antimetabolite selected from aminopterin, methotrexate, pemetrexed, raltitrexed, cladribine, clofarabine, fludarabine, mercaptopurine, pentostatin, thioguanine, capecitabine, cytarabine, 5-fluorouracil, floxuridine and gemcitabine;
(g) an anti-cancer monoclonal antibody selected from alemtuzumab, bevacizumab, cetuximab, gemtuzumab, panitumumab, rituximab, tositumomab and trastuzumab;
(h) a tyrosine kinase inhibitor selected from dasatinib, erlotinib, gefitinib, imatinib, lapatinib, nilotinib, sorafenib and sunitinib; or
(i) combinations thereof.
